(19) United States
(12) Patent Application Publication
(10) Pub. No.: US 2018/0274013 A1
(43) Pub. Date: Sep. 27, 2018

(54) METAL-ORGANIC FRAMEWORK NANOSHEET

(71) Applicant: NANYANG TECHNOLOGICAL UNIVERSITY, Singapore (SG)

(72) Inventors: Hua Zhang, Singapore (SG); Meiting Zhao, Singapore (SG)

(21) Appl. No.: 15/762,328

(22) PCT Filed: Sep. 23, 2016
(86) PCT No.: PCT/SG2016/050471

§ 371 Date: Mar. 22, 2018

(30) Foreign Application Priority Data
Sep. 23, 2015 (SG) 10201507945W

(51) Int. Cl. C12Q 1/6825; B82B 1/00; B82Y 15/00; G01N 33/84; C07D 487/22

(52) U.S. Cl. CPC C12Q 1/6825; B82B 1/007; C07D 487/22; G01N 33/84; B82Y 15/00

(57) ABSTRACT

A method of preparing a metal-organic framework nanosheet is provided. The method includes providing a mixture comprising a metal precursor, a ligand, and a surfactant by at least substantially dissolving the metal precursor, the ligand, and the surfactant in a suitable solvent, and heating the mixture to obtain the metal-organic framework nanosheet. A metal-organic framework nanosheet, methods of preparing a metal-organic framework membrane and a composite material, and applications of the nanosheet and/or membrane in sensing and separation are also provided.

### CROSS-REFERENCE TO RELATED APPLICATION

This application claims the benefit of priority of Singapore patent application No. 10201507945W filed on 23 Sep. 2015, the content of which is incorporated herein by reference in its entirety for all purposes.

### TECHNICAL FIELD

Various embodiments relate to a metal-organic framework nanosheet, a metal-organic framework membrane, their methods of preparation, and application of the metal-organic framework nanosheet and/or membrane in sensing and separation.

### BACKGROUND

Two-dimensional (2D) layered nanomaterials, such as graphene and graphene oxide, transition metal dichalcogenides (TMDs), metal oxides and hydroxides, and boron nitride (BN), have received research interest in recent years due to their unique physical and chemical properties resulting from their ultrathin thickness and 2D morphology.

Inspired by their unique features and applications, considerable efforts have been devoted to exploration of other 2D nanomaterials. Very recently, metal-organic framework (MOF) nanosheets as a new member of the 2D family were successfully prepared.

MOF refers generally to a crystalline porous material constructed by coordination of metal ions or clusters with polytopic organic ligands. It possesses many promising features, such as tunable structure and function, large surface area, and highly ordered pores. In particular, the specific functionality of MOFs may be achieved by changing its constituent metal ions and ligands. Like other 2D materials, MOF nanosheets possess many highly accessible active sites on their surface, which could be significant for applications in catalysis, electrochemistry, and sensing.

Two strategies in the form of top-down and bottom-up methods, may be used to prepare MOF nanosheets. The former involves delamination of bulk MOFs, while the latter may be used to synthesize MOF nanosheets. The top-down method is simple since the usage of sonication or shaking is sufficient to disintegrate the weak interlayer interaction in MOFs. For example, exfoliation of bulk MOFs to form MOF nanosheets have been carried out in water (H₂O), acetone, methanol, ethanol, and tetrahydrofuran.

Unfortunately, this method is not feasible for the uniform and high-yield preparation of MOF nanosheets as the obtained yield is normally less than 15%, and the obtained nanosheets are not stable due to their restacking. In contrast, the bottom-up method is preferred for use in preparing well-dispersed MOF nanosheets in high yield. The bottom-up method, however, is generally carried out by preparing MOF nanosheets or nanofilms on a substrate, and not directly synthesized in solution without the use of a substrate.

In view of the above, there exists a need for an improved method of preparing metal-organic framework nanosheets that overcomes or at least alleviates one or more of the above-mentioned problems.

### SUMMARY

In a first aspect, a method of preparing a metal-organic framework nanosheet is provided. The method comprises
a) providing a mixture comprising a metal precursor, a ligand, and a surfactant by at least substantially dissolving the metal precursor, the ligand, and the surfactant in a suitable solvent, and
b) heating the mixture to obtain the metal-organic framework nanosheet.

In a second aspect, a metal-organic framework nanosheet prepared by a method according to the first aspect is provided.

In a third aspect, a metal-organic framework nanosheet is provided. The metal-organic framework nanosheet has general formula (I)

M₁-L-M₂ (I),

wherein
M₁ is selected from the group consisting of zinc (Zn), copper (Cu), cadmium (Cd), cobalt (Co), zirconium (Zr), aluminum (Al), indium (In), and combinations thereof,
M₂ is nothing or selected from the group consisting of iron (Fe), cobalt (Co), nickel (Ni), manganese (Mn), and combinations thereof,
L is selected from the group consisting of tetrakis(4-carboxyphenyl)porphyrin (TCPP), terephthalic acid (BDC), 2-aminoterephthalic acid (BDC-NH₂), 2,6-naphthalenedicarboxylic acid, 1,3,5-tris(4-carboxyphenyl)benzene (BTB), and combinations thereof,
with the proviso that the metal-organic framework nanosheet is not copper 1,4-benzenedicarboxylate (Cu-BDC).

In a fourth aspect, a metal-organic framework membrane is provided. The metal-organic framework membrane comprises a plurality of metal-organic framework nanosheets according to the third aspect.

In a fifth aspect, a method of preparing a metal-organic framework membrane is provided. The method comprises
a) preparing one or more metal-organic framework nanosheets according to the method of the first aspect or providing one or more metal-organic framework nanosheets according to the third aspect,
b) dispersing the one or more metal-organic framework nanosheets in an aqueous solution to form a mixture, and
c) filtering the mixture through a membrane such that the one or more metal-organic framework nanosheets is deposited on the membrane to obtain the metal-organic framework membrane.

In a sixth aspect, a method of preparing a composite material comprising a metal-organic framework nanosheet and a noble metal nanoparticle is provided. The method comprises
a) preparing a metal-organic framework nanosheet according to the method of the first aspect or providing a metal-organic framework nanosheet according to the third aspect, and
b) dispersing the metal-organic framework nanosheet in an aqueous solution comprising a noble metal nanoparticle precursor and a reducing agent to obtain the composite material.

In a seventh aspect, use of a metal-organic framework nanosheet prepared by a method according to the first aspect or a metal-organic framework nanosheet according to the third aspect in sensing, preferably for detecting DNA and/or for detecting hydrogen peroxide (H₂O₂) is provided.

In an eighth aspect, use of a metal-organic framework membrane according to the fourth aspect or prepared by a method according to the fifth aspect in separation, preferably for separating organic dyes of different sizes, is provided.

### BRIEF DESCRIPTION OF THE DRAWINGS

The invention will be better understood with reference to the detailed description when considered in conjunction with the non-limiting examples and the accompanying drawings, in which:

FIG. 1 is a schematic diagram showing traditional synthesis and surfactant-assisted synthesis of MOF. Top: During synthesis of MOFs in a traditional method, the isotropic growth generates the bulk crystal of MOFs. Bottom: By using the developed surfactant-assisted synthetic method disclosed herein, selective attachment of surfactants on the surface of MOFs leads to their anisotropic growth, resulting in the formation of ultrathin MOF nanosheets. The MOF layers are shown in different shades (indicated ‘Blue MOF layer” and “Purple MOF layer”) to make the layered structures clear.

FIG. 2A is a scanning transmission electron microscopy (STEM) image of zinc-tetrakis(4-carboxyphenyl)porphyrin (Zn-TCPP) nanosheets using scanning electron microscopy (SEM) with a transmission electron detector (inset: Tyndall effect of colloidal Zn-TCPP nanosheet in ethanol).

FIG. 2B is a transmission electron microscopy (TEM) image of a single Zn-TCPP nanosheet.

FIG. 2C is an Atomic Force Microscopy (AFM) image of Zn-TCPP nanosheets. Scale bar: 2 μm.

FIG. 2D is a high-resolution transmission electron microscopy (HRTEM) image of a Zn-TCPP nanosheet and corresponding fast Fourier transform (FFT) pattern (inset).

FIG. 2E is a selected-area electron diffraction (SAED) pattern of a Zn-TCPP nanosheet.

FIG. 2F is a X-ray powder diffraction (XRD) pattern of (i) Zn-TCPP nanosheets and (ii) bulk Zn-TCPP material.

FIG. 3A shows nitrogen (N₂) adsorption-desorption isotherms of Zn-TCPP nanosheets and bulk Zn-TCPP MOFs.

FIG. 3B shows corresponding pore-size distribution curves of (i) Zn-TCPP nanosheets and (ii) bulk Zn-TCPP MOFs.

FIG. 4 shows Fourier transform infrared (FTIR) spectra of bulk Zn-TCPP MOFs, Zn-TCPP nanosheets, Zn(NO₃)₂, polyvinylpyrrolidone (PVP) and the mixture of zinc nitrate (Zn(NO₃)₂) and PVP with a mole ratio of 1:1.

FIG. 5A shows a STEM image of Cu-TCPP nanosheets. Insets: TEM images and corresponding SAED patterns of the MOF nanosheets.

FIG. 5B shows a STEM image of Cd-TCPP nanosheets. Insets: TEM images and corresponding SAED patterns of the MOF nanosheets.

FIG. 5C shows a STEM image of Co-TCPP nanosheets. Insets: TEM images and corresponding SAED patterns of the MOF nanosheets.

FIG. 5D shows a STEM image of Zn-TCPP(Fe) nanosheets. Insets: TEM images and corresponding SAED patterns of the MOF nanosheets.

FIG. 5E shows a STEM image of Cu-TCPP(Fe) nanosheets. Insets: TEM images and corresponding SAED patterns of the MOF nanosheets.

FIG. 5F shows a STEM image of Co-TCPP(Fe) nanosheets. Insets: TEM images and corresponding SAED patterns of the MOF nanosheets.

FIG. 6A shows AFM image of Cu-TCPP nanosheets. Scale bar: 2 μm. The average thickness of Cu-TCPP nanosheets obtained from AFM images was 4.5±1.2 nm.

FIG. 6B shows AFM image of Cd-TCPP nanosheets. Scale bar: 2 μm. The average thickness of Cd-TCPP nanosheets obtained from AFM images was 8.7±2.7 nm.

FIG. 6C shows AFM image of Co-TCPP nanosheets. Scale bar: 2 μm. The average thickness of Co-TCPP nanosheets obtained from AFM images was 18.8±6.4 nm.

FIG. 6D shows AFM image of 2D MOF nanosheets of Zn-TCPP(Fe) nanosheets. Scale bar: 2 μm. The average thickness of Zn-TCPP(Fe) nanosheets obtained from AFM images was 7.4±2.9 nm.

FIG. 7A is a SEM image of bulk Cu-TCPP MOFs which do not have the nanosheet structure.

FIG. 7B is a SEM image of Cu-TCPP nanosheets.

FIG. 7C shows powder XRD patterns of (i) Cu-TCPP nanosheets and (ii) bulk Cu-TCPP MOFs depicted in FIG. 7A and FIG. 7B. Prior to XRD characterization, the samples were dried at 100° C. for 2 h.

FIG. 7D is a SEM image of bulk Cd-TCPP MOFs which do not have the nanosheet structure.

FIG. 7E is a SEM image of Cd-TCPP nanosheets.

FIG. 7F shows powder XRD patterns of (i) Cd-TCPP nanosheets and (ii) bulk Cd-TCPP MOFs depicted in FIG. 7D and FIG. 7E. Prior to XRD characterization, the samples were dried at 100° C. for 2 h.

FIG. 7G is a SEM image of bulk Co-TCPP MOFs which do not have the nanosheet structure.

FIG. 7H is a SEM image of Co-TCPP nanosheets.

FIG. 7I shows powder XRD patterns of (i) Co-TCPP nanosheets and (ii) bulk Co-TCPP MOFs depicted in FIG. 7G and FIG. 7H. Prior to XRD characterization, the samples were dried at 100° C. for 2 h.

FIG. 7J is a SEM image of bulk Zn-TCPP(Fe) MOFs which do not have the nanosheet structure.

FIG. 7K is a SEM image of Zn-TCPP(Fe) nanosheets.

FIG. 7L shows powder XRD patterns of (i) Zn-TCPP(Fe) nanosheets and (ii) bulk Zn-TCPP(Fe) MOFs depicted in FIG. 7J and FIG. 7K. Prior to XRD characterization, the samples were dried at 100° C. for 2 h.

FIG. 8A is a TEM image of Cu-TCPP(Co) nanosheets.

FIG. 8B is a magnified TEM image of Cu-TCPP(Co) nanosheets.

FIG. 8C is a TEM image of Cu-TCPP(Mn) nanosheets.

FIG. 8D is a magnified TEM image of Cu-TCPP(Mn) nanosheets.

FIG. 9A is a SEM image of Al-TCPP nanosheets.

FIG. 9B is a SEM image of Cu-BDC nanosheets.

FIG. 9C is a SEM image of Cu-BDC-NH₂ nanosheets.

FIG. 9D is a SEM image of Al-BDC nanosheets.

FIG. 9E is a SEM image of Al-BDC-NH₂ nanosheets.

FIG. 10 is a graph showing fluorescence spectra of Cu-TCPP nanosheets, Zn-TCPP(Fe) nanosheets, Co-TCPP nanosheets, Cd-TCPP nanosheets and Zn-TCPP nanosheets. Excitation wavelength: 420 nm.

FIG. 11A is a schematic illustration of MOF nanosheet-based fluorescent DNA assay, depicting DNA detection with 2D MOF (Cu-TCPP, Zn-TCPP(Fe), Co-TCPP).

FIG. 11B is a graph showing fluorescence spectra at different experimental conditions: (I) P1; (II) P1+T1+Cu-TCPP nanosheets; (III) P1+Cu-TCPP nanosheets; and (IV) Cu-TCPP nanosheets. The concentrations of P1, T2 and Cu-TCPP nanosheet in the final solution are 2.5 nM, 20 nM and 35 μg/mL, respectively. Inset: Kinetic study on the fluorescence change of P1 and P1/T1 duplex in the presence of Cu-TCPP nanosheets. Excitation and emission wavelengths are 588 and 609 nm, respectively.

FIG. 11C shows the quenching efficiency (η) of Cu-TCPP nanosheet and bulk Cu-TCPP MOFs for P1 and P1/T1 (left); and the fluorescence intensity ratio (F_{P1/T1}/F_{P1}) at 609 nm in the presence of Cu-TCPP nanosheets (35 μg mL⁻¹) and bulk Cu-TCPP MOFs (35 μg mL⁻¹) (right). F_{P1/T1} and F_{P1} are the fluorescence intensity of ds DNA (P1/T1) and ssDNA (P1) at 609 nm in the presence of Cu-TCPP nanosheets and bulk Cu-TCPP MOFs, respectively. The concentrations of P1 and T1 in the final solution are 2.5 nM and 20 nM.

FIG. 11D is a graph showing fluorescence spectra of P1 (2.5 nM) in the presence of T1 with different concentrations in Cu-TCPP nanosheet solution (35 μg mL⁻¹).

FIG. 12A is a graph showing fluorescence spectra recorded at experimental conditions of P1, P1+T1+Zn-TCPP(Fe) nanosheets, P1+Zn-TCPP(Fe) nanosheets, and Zn-TCPP(Fe) nanosheets.

FIG. 12B is a graph showing fluorescence spectra recorded at experimental conditions of P1, P1+T1+bulk Zn-TCPP(Fe) MOFs, P1+bulk Zn-TCPP(Fe) MOFs and bulk Zn-TCPP(Fe) MOFs.

FIG. 12C is a graph showing fluorescence spectra recorded at experimental conditions of P1, P1+T1+Co-TCPP nanosheets, P1+Co-TCPP nanosheets, and Co-TCPP nanosheets.

FIG. 12D is a graph showing fluorescence spectra recorded at experimental conditions of P1, P1+T1+bulk Co-TCPP MOFs, P1+bulk Co-TCPP MOFs, and bulk Co-TCPP MOFs.

FIG. 12E is a graph showing quenching efficiency (η) of Zn-TCPP(Fe) nanosheets, bulk Zn-TCPP(Fe) MOFs, Co-TCPP nanosheets and bulk Co-TCPP MOFs for P1 and P1/T1.

FIG. 12F is a graph showing fluorescence intensity ratio (F_{P1/T1}/F_{P1}) at 609 nm in the presence of Zn-TCPP(Fe) nanosheets, bulk Zn-TCPP(Fe) MOFs, Co-TCPP nanosheets and bulk Co-TCPP MOFs. The concentrations of P1, T1 and MOF (Zn-TCPP(Fe) nanosheets, or bulk Zn-TCPP(Fe) MOFs, or Co-TCPP nanosheets, or bulk Co-TCPP MOFs) in the final solution are 2.5 nM, 20 nM and 35 μg mL⁻¹, respectively. Excitation and emission wavelengths are 588 and 609 nm, respectively.

FIG. 13A is a graph showing fluorescence spectra for multiplexed detection using 2D Cu-TCPP nanosheets (35 μg mL⁻¹). Probe mixture (P1+P2) in the absence of T1 and T2. Curves I and II correspond to fluorescence signal of P1 and P2, with different excitation and emission wavelengths of 588 nm/609 nm and 522 nm/539 nm, respectively. The concentrations of probe (P1 and P2) and target DNA (T1 and T2) in the final solution are 2.5 nM and 20 nM, respectively.

FIG. 13B is a graph showing fluorescence spectra for multiplexed detection using Cu-TCPP nanosheets (35 μg mL⁻¹). Probe mixture (P1+P2) in the presence of T1 and absence of T2. Curves I and II correspond to fluorescence signal of P1 and P2, with different excitation and emission wavelengths of 588 nm/609 nm and 522 nm/539 nm, respectively. The concentrations of probe (P1 and P2) and target DNA (T1 and T2) in the final solution are 2.5 nM and 20 nM, respectively.

FIG. 13C is a graph showing fluorescence spectra for multiplexed detection using Cu-TCPP nanosheets (35 μg mL⁻¹). Probe mixture (P1+P2) in the presence of T2 and absence of T1. Curves I and II correspond to fluorescence signal of P1 and P2, with different excitation and emission wavelengths of 588 nm/609 nm and 522 nm/539 nm, respectively. The concentrations of probe (P1 and P2) and target DNA (T1 and T2) in the final solution are 2.5 nM and 20 nM, respectively.

FIG. 13D is a graph showing fluorescence spectra for multiplexed detection using Cu-TCPP nanosheets (35 μg mL⁻¹). Probe mixture (P1+P2) in the presence of T1 and T2. Curves I and II correspond to fluorescence signal of P1 and P2, with different excitation and emission wavelengths of 588 nm/609 nm and 522 nm/539 nm, respectively. The concentrations of probe (P1 and P2) and target DNA (T1 and T2) in the final solution are 2.5 nM and 20 nM, respectively.

FIG. 14 is a graph showing selective detection with P1 (left panel) and P2 (right panel) using Cu-TCPP nanosheets: complementary target DNA (T1 and T2), single-base mismatch DNA (SM1 and SM2), random DNA (R) and blank (B). The concentrations of probe (P1 and P2), target DNA (T1 and T2), single-base mismatch DNA (SM1 and SM2), random DNA (R) and Cu-TCPP nanosheets in the final solution are 2.5 nM, 20 nM, 20 nM, 20 nM and 35 μg mL⁻¹, respectively.

FIG. 15A is a graph showing cyclic voltammetry (CV) curves of glass carbon electrode (GCE), horseradish peroxidase modified glass carbon electrode (HRP/GCE) and 2D Co-TCPP(Fe)/GCE in 0.1 M PBS (pH 7.4) containing 0.5 m M H₂O₂. Scan rate: 50 mV s⁻¹.

FIG. 15B is a graph showing typical amperometric response of 2D Co-TCPP(Fe)/GCE and HRP/GCE to successive addition of different H₂O₂ concentration in 0.1 M PBS (pH 7.4).

FIG. 15C is a graph showing calibration curve of the 2D Co-TCPP(Fe)/GCE corresponding to amperometric response at −50 mV.

FIG. 15D is a graph showing calibration curve of the 2D Co-TCPP(Fe)/GCE corresponding to amperometric response at −50 mV.

FIG. 15E shows amperometric responses of 2D Co-TCPP(Fe)/GCE to the analytes of interest at different detection potential.

FIG. 15F shows amperometric responses of the 2D Co-TCPP(Fe)/GCE in 0.1 M PBS (pH 7.4) with the addition of 10 μM fMLP and 300 U mL⁻¹ catalase in the absence (upper) and present (bottom) of cells. Inset: the bright-field microscopy image of human breast adenocarcinoma cells (MDA MB 231).

FIG. 16A is a SEM image of the surface of MOF membrane prepared from ultrathin Cu-TCPP nanosheets. Scale bar denotes 1 μm.

FIG. 16B is a SEM image of the cross-section of MOF membrane prepared from ultrathin Cu-TCPP nanosheets. Scale bar denotes 100 nm.

FIG. 16C shows separation of methyl orange (orange) and Brilliant blue G 250 (blue) by filtration using MOF membrane. The mixed solution was forced through a MOF membrane (25 mm diameter), allowing permeation of methyl orange only, while retaining Brilliant blue G 250. Concentrations of methyl orange and Brilliant blue G 250 were 20 PPM.

FIG. 17A is a SEM image of MOF heterostructures of MIL-69/Al-TCPP. Scale bar denotes 1 μm.

FIG. 17B is a SEM image of MOF heterostructures of MIL-69/Al-TCPP. Scale bar denotes 100 nm.

FIG. 17C is a SEM image of MOF heterostructures of MIL-68/In-TCPP. Scale bar denotes 1 μm.

FIG. 17D is a SEM image of MOF heterostructures of MIL-68/In-TCPP. Scale bar denotes 1 μm.

FIG. 17E is a SEM image of MOF heterostructures of MOF-525/Zr-BTB. Scale bar denotes 1 μm.

FIG. 17F is a SEM image of MOF heterostructures of MOF-525/Zr-BTB. Scale bar denotes 1 μm.

FIG. 18A is a schematic illustration of oxidation of 3,3′,5,5′-tetramethylbenzidine (TMB) catalyzed by MOF nanosheets-based catalyst.

FIG. 18B is a time-dependent absorption spectra of TMB in the presence of H₂O₂ and Cu-TCPP(Fe) nanosheets. Inset: photograph.

FIG. 18C is a time-dependent absorption spectra of TMB in the presence of H₂O₂ and Cu-TCPP(Co) nanosheets. Inset: photograph.

FIG. 18D is a time-dependent absorption spectra of TMB in the presence of H₂O₂ and Cu-TCPP(Mn) nanosheets. Inset: photograph.

FIG. 19A is a graph showing UV-vis absorbance spectra of TMB in different reaction systems: TMB (purple line), TMB+H₂O₂ (orange line), TMB+H₂O₂+Cu-TCPP(Fe) (green line) in HAc-NaAc buffer solution (10 mM, pH 4.0) containing 800 μM TMB, 1 mM H₂O₂ and 20 μg mL⁻¹ Cu-TCPP(Fe) nanosheets at room temperature.

FIG. 19B is a graph showing UV-vis absorbance spectra of TMB in different reaction systems: TMB (purple line), TMB+H₂O₂ (orange line), TMB+H₂O₂+Cu-TCPP(Co) (green line) in HAc-NaAc buffer solution (10 mM, pH 4.0) containing 800 μM TMB, 5 mM H₂O₂ and 30 μg mL⁻¹ Cu-TCPP(Co) nanosheets at room temperature.

FIG. 19C is a graph showing UV-vis absorbance spectra of TMB in different reaction systems: TMB (purple line), TMB+H₂O₂ (orange line), TMB+H₂O₂+Cu-TCPP(Mn) (green line) in HAc-NaAc buffer solution (10 mM, pH 4.0) containing 800 μM TMB, 15 mM H₂O₂ and 30 μg mL⁻¹ Cu-TCPP(Mn) nanosheets at room temperature.

FIG. 20A is a TEM image of Au/Cu-TCPP(Fe) hybrid nanomaterial.

FIG. 20B is a magnified TEM image of Au/Cu-TCPP(Fe) hybrid nanomaterial.

FIG. 20C is a HRTEM image of Au NPs on Cu-TCPP(Fe).

FIG. 20D is a SAED pattern of Au NPs on Cu-TCPP(Fe).

FIG. 20E is a schematic illustration of cascade catalysis based on Cu-TCPP(Fe)—Au hybrid nanomaterial.

FIG. 20F is a graph showing UV-vis absorbance spectra of TMB in different reaction systems.

FIG. 21A is a graph showing UV-vis absorbance spectra of TMB in different reaction systems: TMB (orange line), TMB+H₂O₂ (dark yellow line), TMB+H₂O₂+Au/Cu-TCPP(Fe) (green line) in 800 μM TMB solution containing 1 mM H₂O₂ and 30 μg mL⁻¹ 2 D Au/Cu-TCPP(Fe) at room temperature.

FIG. 21B is a graph showing UV-vis absorbance spectra for different samples obtained by a gluconic acid-specific assay: only glucose (orange line), Au/Cu-TCPP(Fe) alone (dark yellow line), glucose+Au/Cu-TCPP(Fe) (green line).

### DETAILED DESCRIPTION

As disclosed herein, a bottom-up synthesis method for the preparation of ultrathin metal-organic frameworks (MOFs) nanosheets is provided. The term “MOF nanosheet”, which is used exchangeably with the term “two-dimensional (2D) MOF”, refers to a MOF having a two-dimensional sheet-like structure. The MOF nanosheet may be planar, and have a thickness of less than 20 nm, such as less than 15 nm or less than 10 nm, while having lateral dimensions of 0.7 μm or more, such as 1 μm or more, or ranging from hundreds of nanometers to tens of micrometers. Through the use of surfactants which selectively attach on a surface of MOFs, stacking of the MOF layered sheets may be restricted to result in formation of ultrathin MOF nanosheets.

Taking PVP assisted synthesis of Zn-TCPP nanosheets as an example, C═O group in PVP was shown herein to interact strongly with Zn²⁺ ions, as can be seen by shifting of the stretching vibration of ν_{C═O} in PVP at 1662 cm⁻¹ to 1619 cm⁻¹ after mixing with Zn(NO₃)₂. Therefore, PVP may attach on a surface of MOF through interaction of C═O group with metal nodes, thereby restricting stacking of the layered sheets and form ultrathin Zn-TCPP nanosheets.

Notably, the MOF nanosheets prepared using a method according to embodiments disclosed herein are freestanding, as they may be directly synthesized in solution without the use of a supporting substrate. Examples of freestanding ultrathin MOF nanosheets which may be prepared by methods disclosed herein include, but are not limited to, M-TCPP (M=Zn, Cu, Cd, Co, Zr, Al, or In) nanosheets, M-BDC (M=Cu, Al) and M-BDC-NH₂ (M=Cu, Al) nanosheets. Methods disclosed herein may also be extended to hetero-metal MOF nanosheets-containing functional groups, such as M-TCPP(M′), where M includes, but are not limited to, Zn, Cu, Cd, Co, Zr, Al, or In, and M′ includes but are not limited to Fe, Co, Ni and Mn.

In alternative embodiments, the MOF nanosheets may be prepared on one-dimensional MOF nanostructured materials, such as nanoribbons, nanorods, and/or nanospheres, to form MOF heterostructures. The MOF nanosheets may also be used to form a composite with a noble metal nanoparticle such as a gold nanoparticle. In various embodiments, the MOF nanosheets may also be used to form a MOF membrane.

Advantageously, due to the facile preparation procedures, both freestanding MOF nanosheets and MOF heterostructures may be prepared easily with high yield using the surfactant-assisted bottom-up synthesis method disclosed herein. As compared with other bottom-up synthesis methods, there is greater ease in preparing ultrathin MOF nanosheets without their restacking due to protection role of surfactants. There is much improved yield and greater ease in synthesizing freestanding MOF nanosheets as compared to top-down methods. As mentioned above, lateral size of the metal-organic framework nanosheet may be about 0.7 μm or more, such as 1 μm or more, while thickness of the metal-organic framework nanosheet may be about 10 nm or less, which renders the metal-organic framework nanosheet “ultrathin”.

In relation to bulk MOF material, the ultrathin MOF nanosheets with their higher surface area exposes more active sites on their surface, which may be significant for applications in sensing and separation. The high-throughput and facile fabrication process as well as the scalable method means that the methods disclosed herein may be adapted readily for industrial usage.

With the above in mind, various embodiments refer in a first aspect to a method of preparing a metal-organic framework nanosheet. The method comprises providing a mixture comprising a metal precursor, a ligand, and a surfactant by at least substantially dissolving the metal precursor, the ligand, and the surfactant in a suitable solvent, and heating the mixture to obtain the metal-organic framework nanosheet.

The metal precursor may be a M(II) or M(III) salt or complex, where M is a metal. Generally, any metal precursor that may be at least substantially dissolved in a suitable solvent along with the ligand and the surfactant may be used. In some embodiments, the metal precursor is selected from the group consisting of metal nitrate, metal chloride, metal acetate, metal sulfate, and combinations thereof. In specific embodiments, the metal precursor is metal nitrate and/or metal chloride.

Metal of the metal precursor may generally be selected from Period 4 or Period 5 of d-block element, or Group 13 of the Periodic Table of elements, where they may form a divalent metal ion or a trivalent metal ion. In various embodiments, the metal of the metal precursor is a transition metal and/or a Group 13 metal. Examples of a transition metal include scandium (Sc), titanium (Ti), vanadium (V), chromium (Cr), manganese (Mn), iron (Fe), cobalt (Co), nickel (Ni), copper (Cu), zinc (Zn), zirconium (Zr), cadmium (Cd), and alloys thereof. Examples of a Group 13 metal include boron, aluminum, gallium, indium and thallium. In some embodiments, metal of the metal precursor is selected from the group consisting of zinc, copper, cadmium, cobalt, zirconium, aluminum, indium, and combinations thereof.

As used herein, the term “ligand” refers to a molecule, such as an organic ligand compound containing one or more functional groups, or a functional group that is able to bind to a central metal atom to form a coordination complex. Examples of ligands may include without limitation, tetrakis(4-carboxyphenyl)porphyrin (TCPP), TCPP(M′), wherein M′ is a metal different from the metal of the metal precursor and is selected from the group consisting of Fe, Co, Ni, Mn, and combinations thereof, terephthalic acid, 2-aminoterephthalic acid (BDC-NH₂), 2,6-naphthalenedicarboxylic acid, 1,3,5-tris(4-carboxyphenyl)benzene (BTB), biphenyl-dicarboxylic acid, benzene tricarboxylic, di(carboxyphenyl)benzene, imidazole, benzimidazole, and alkane, alkene and alkyne dicarboxylic acids.

In various embodiments, the ligand is selected from the group consisting of tetrakis(4-carboxyphenyl)porphyrin (TCPP), TCPP(M′), wherein M′ is a metal different from the metal of the metal precursor and is selected from the group consisting of Fe, Co, Ni, Mn, and combinations thereof, terephthalic acid (BDC), 2-aminoterephthalic acid (BDC-NH₂), 2,6-naphthalenedicarboxylic acid, 1,3,5-tris(4-carboxyphenyl)benzene (BTB), and combinations of the above-mentioned.

Besides the metal precursor and the ligand, the mixture also contains a surfactant. As used herein, the term “surfactant” refers to materials that have an amphiphilic molecular structure, which may include a polar hydrophilic molecular moiety and a nonpolar lipophilic molecular moiety. As mentioned above, selective attachment of surfactants on the surface of MOFs may restrict stacking of the layered MOF sheets to result in their anisotropic growth and formation of ultrathin MOF nanosheets. The surfactant may selectively attach to the MOF nanosheets via interaction of one or more functional groups such as C═O group on the surfactant with metal nodes of the MOF nanosheets.

The surfactant may, for example, be selected from the group consisting of cetyltrimethylammonium bromide (CTAB), cetyltrimethylammonium chloride (CTAC), polyvinylpyrrolidone (PVP), polyethylene glycol (PEG), sodium dodecyl sulfate (SDS), and combinations thereof.

In specific embodiments, the surfactant is cetyltrimethylammonium bromide (CTAB), polyvinylpyrrolidone (PVP), or combinations thereof.

The mixture comprising the metal precursor, the ligand, and the surfactant is provided by at least substantially dissolving the metal precursor, the ligand, and the surfactant in a suitable solvent. The term “substantially dissolving” as used herein refers to cases where at least 75 wt %, at least 80 wt %, at least 85 wt %, at least 90 wt %, at least 95 wt %, or at least 99 wt % of a material is dissolved in a solvent. By substantially dissolving the metal precursor, the ligand, and the surfactant in a suitable solvent, microemulsions are not formed. In other words, method of preparing a metal-organic framework nanosheet according to embodiments disclosed herein does not involve formation of a microemulsion.

In various embodiments, the solvent is selected from the group consisting of N,N-dimethylformamide (DMF), dimethylacetamide (DMA), N,N-diethylformamide (DEF), ethanol, methanol, water, and combinations thereof.

In specific embodiments, the solvent is a mixture of N,N-dimethylformamide and ethanol with a volumetric ratio in the range from about 1:1 to about 4:1.

Providing the mixture may comprise adding the ligand to a solution comprising the metal precursor and the surfactant, or by adding the surfactant and the ligand to an aqueous solution comprising the metal precursor.

In embodiments where the metal of the metal precursor is a transition metal, for example, providing the mixture may comprise adding the ligand to a solution comprising the metal precursor and the surfactant. In various embodiments, the ligand is dissolved in a mixture of N,N-dimethylformamide and ethanol with a volumetric ratio in the range from about 1:1 to about 4:1, such as about 3:1. Adding the ligand to the solution may be carried out in a drop wise manner while the solution is being physically agitated, such as by stirring or sonication.

In embodiments where the metal of the metal precursor is a Group 13 metal such as aluminum or indium, providing the mixture comprises adding the surfactant and the ligand to an aqueous solution comprising the metal precursor. In various embodiments, the surfactant and the ligand is dissolved in ethanol. Adding the surfactant and the ligand to the aqueous solution comprising the metal precursor is carried out in a drop wise manner while the aqueous solution comprising the metal precursor is being physically agitated.

In addition to the metal precursor, the ligand and the surfactant, the mixture may further comprise a crystallinity enhancing agent, a ligand dissolution enhancing agent, and/or a growth control agent. One or more of the crystallinity enhancing agent, ligand dissolution enhancing agent, or growth control agent may be added to the mixture comprising the metal precursor, the ligand and the surfactant prior to heating.

The crystallinity enhancing agent may be added, for example, to increase crystallinity of the metal-organic framework nanosheet formed so as to obtain high crystallinity MOFs with highly ordered pores. In various embodiments, the crystallinity enhancing agent is selected from the group consisting of pyrazine, 4,4′-bipyridyl, and combinations thereof. In specific embodiments, the crystallinity enhancing agent is pyrazine.

The ligand dissolution enhancing agent, on the other hand, may be added to improve the rate at which the ligand is dissolved in the mixture. In various embodiments, the ligand dissolution enhancing agent is an alkali. In some embodiments, the ligand dissolution enhancing agent is selected from the group consisting of pyridine, ammonia, trimethylamine, ethylenediamine, an alkali metal hydroxide such as sodium hydroxide and potassium hydroxide, and combinations thereof.

The growth control agent may be added to reduce the growth rate of MOF nanosheets, due to competition between the growth control agent and the MOF ligands. As a result of the competition, size and thickness of the MOFs being formed may be controlled and/or reduced to favor formation of MOF nanosheets. In various embodiments, the growth control agent is an acid. In some embodiments, the growth control agent is selected from the group consisting of trifluoroacetic acid, acetic acid, dichloroacetic acid, formic acid, benzoic acid, and combinations thereof.

The mixture comprising the metal precursor, the ligand, and the surfactant is heated to obtain the metal-organic framework nanosheet. Prior to heating, the mixture may be stirred or sonicated for a time period in the range of about 5 minutes to about 15 minutes, such as about 5 minutes to about 10 minutes, about 8 minutes to about 12 minutes, or about 10 minutes. The stirring or sonication may be carried out to improve on dispersion or mixing of the metal precursor, the ligand, and the surfactant in the mixture.

Heating the mixture may be carried out at a temperature in the range of about 60° C. to about 160° C., such as about 60° C. to about 100° C., about 60° C. to about 80° C., about 80° C. to about 100° C., about 100° C. to about 160° C., or about 120° C. to about 160° C., and may be carried for a time period sufficient to form the MOF nanosheets, such as a time period in the range of about 3 hours to about 24 hours, such as about 8 hours to about 24 hours, about 10 hours to about 24 hours, about 3 hours to about 20 hours, about 3 hours to about 18 hours, about 3 hours to about 10 hours, about 10 hours to about 20 hours, or about 8 hours to about 18 hours.

In various embodiments, heating the mixture is carried out under hydrothermal conditions, and may for example, be carried out in an autoclave. This may, for example, be used for preparing Group 13 metal-organic framework nanosheets such as aluminum metal-organic framework nanosheets.

As mentioned above, the MOF nanosheets may be prepared on one-dimensional MOF nanostructured materials, such as nanoribbons, nanorods, and/or nanospheres, to form MOF heterostructures. Accordingly, the method of the first aspect may include at least substantially dissolving the metal precursor, the ligand, and the surfactant in a suitable solvent in the presence of a metal-organic framework nanostructured material. The metal-organic framework nanostructured material may be prepared using a method to form MOF nanosheets disclosed herein, but in the absence of a surfactant in the mixture. In the absence of a surfactant, one dimensional MOF nanostructured materials such as nanoribbons, nanorods, and/or nanospheres may be formed. In such MOF heterostructures, the metal-organic framework nanostructured material may comprise a metal identical to the metal of the metal precursor. As demonstrated herein, MOF heterostructures comprising MOF nanosheets grown on one dimensional MOF nanostructures have been obtained.

Various embodiments refer in a second aspect to a metal-organic framework nanosheet prepared by a method according to the first aspect, and in a third aspect to a metal-organic framework nanosheet having general formula (I)

M₁-L-M₂ (I),

wherein M₁ is selected from the group consisting of zinc (Zn), copper (Cu), cadmium (Cd), cobalt (Co), aluminum (Al), indium (In), and combinations thereof, M₂ is nothing or selected from the group consisting of iron (Fe), cobalt (Co), nickel (Ni), manganese (Mn), and combinations thereof, L is selected from the group consisting of tetrakis(4-carboxyphenyl)porphyrin (TCPP), terephthalic acid (BDC), 2-aminoterephthalic acid (BDC-NH₂), 2,6-naphthalenedicarboxylic acid, 1,3,5-tris(4-carboxyphenyl)benzene (BTB), and combinations thereof, with the proviso that the metal-organic framework nanosheet is not copper 1,4-benzenedicarboxylate (Cu-BDC).

In specific embodiments, the metal-organic framework nanosheet is selected from the group consisting of Zn-TCPP, Cu-TCPP, Cd-TCPP, Co-TCPP, Al-TCPP, In-TCPP, Zr-TCPP, Zn-TCPP-Fe, Cu-TCPP-Fe, Co-TCPP-Fe, Cu-TCPP-Co, Cu-TCPP-Mn, Cu-BDC-NH₂, Al-BDC, Al-BDC-NH₂, and combinations thereof.

As mentioned above, thickness of the metal-organic framework nanosheet may be less than about 10 nm, such as less than about 8 nm, or less than about 5 nm, while lateral size of the metal-organic framework nanosheet may be about 0.7 μm or more, such as 1 μm or more.

The obtained ultrathin 2D MOF nanosheets may be freestanding, and may furthermore be well-dispersed in a wide range of solvents, such as water, methanol, ethanol and acetone, which benefits their usage in sensing applications. Various embodiments thus refer in a further aspect to use of a metal-organic framework nanosheet prepared by a method according to the first aspect or a metal-organic framework nanosheet according to the third aspect in sensing, such as for detecting DNA and/or for detecting hydrogen peroxide (H₂O₂). Details of how the sensing and detection may be carried out are provided in the examples. In particular, the ultrathin MOF nanosheets prepared by a method disclosed herein may serve as new platforms for DNA detection. For example, the Cu-TCPP nanosheet-based sensor disclosed herein has demonstrated excellent fluorescent sensing performance, excellent selectivity, and allows simultaneous detection of multiple DNA targets.

In various embodiments, when the MOF nanosheet is Cu-TCPP for example, the TCPP ligand containing conjugated π-electron system allows for the binding of single-stranded DNA (ssDNA). A dye-labeled ssDNA probe may therefore be adsorbed on the basal surface of MOF nanosheets, resulting in the fluorescence quenching of the dye through the fluorescence resonance energy transfer (FRET). However, when the dye-labeled ssDNA probe is hybridized with its target complementary DNA to form double-stranded DNA (dsDNA), interaction between the formed dsDNA and MOF nanosheet may become so weak that the dsDNA detaches from the surface of MOF nanosheet, resulting in the recovery of quenched fluorescence of dye to provide quantitative detection of the target complementary DNA.

The results obtained shows that the 2D MOF nanosheets could quench many different dyes with distinct absorption/emission properties. This is essential for the proposed development of a multiplex detection platform (i.e. simultaneous detection of several targets), which holds great promise in practical applications.

For detecting hydrogen peroxide (H₂O₂), an electrochemical sensor based on MOF nanosheets such as Co-TCPP(Fe) nanosheets may be used. The MOF nanosheets may be used to modify an electrode such as glass carbon electrode, and used in electrochemical measurements to generate cyclic voltammetry (CV) curves and/or monitor changes in current over time. By observing changes in the generated CV curve or amperometric response, presence of H₂O₂ may be detected. Advantageously, the electrochemical sensor based on MOF nanosheets according to embodiments disclosed herein has exhibited higher sensitivity and better stability than natural enzyme (HRP, horseradish peroxidase), and may be used for real-time detection of H₂O₂ secretion by living cells due to its high sensitivity, fast response time, long-term stability and reproducibility.

The as-synthesized ultrathin 2D MOF nanosheets may also be used as building blocks for the preparation of a composite or a hybrid nanosheet with noble metal nanoparticles. Various embodiments therefore refer in a further aspect to a method of preparing a composite material comprising a metal-organic framework nanosheet and a noble metal nanoparticle.

The method comprises preparing a metal-organic framework nanosheet according to the method of the first aspect or providing a metal-organic framework nanosheet according to the third aspect, and dispersing the metal-organic framework nanosheet in an aqueous solution comprising a noble metal particle precursor and a reducing agent to obtain the composite material. The reducing agent may reduce the noble metal particle precursor to form a noble metal particle, therefore by dispersing the metal-organic framework nanosheet in an aqueous solution comprising the noble metal particle precursor and the reducing agent, a composite material comprising the metal-organic framework nanosheet and the noble metal nanoparticle may be obtained.

In various embodiments, the noble metal nanoparticle is a gold nanoparticle. Accordingly, the noble metal particle precursor may be chloroauric acid (HAuCl₄), while the reducing agent may be sodium borohydride (NaBH₄) and/or hydrazine hydrate.

In embodiments disclosed herein, a hybrid nanosheet based on Cu-TCPP(Fe) and gold nanoparticles (Au NPs) were prepared by in situ growth of Au NPs on Cu-TCPP(Fe) nanosheets. The obtained Au/Cu-TCPP(Fe) hybrid nanosheets may be used to mimic enzyme cascade reaction, in which the Au NPs and Cu-TCPP(Fe) nanosheets possess intrinsic glucose oxidase (Gox)- and peroxidase-like activity, respectively, and may be used in biomimetic catalysis such as that illustrated in the examples. An artificial enzymatic cascade system may therefore be engineered based on 2D Cu-TCPP(M₂) supported Au NPs (Au/Cu-TCPP(M₂)), where suitable M₂ have already been described above.

Apart from the above-mentioned, the as-synthesized ultrathin 2D MOF nanosheets may also be used as building blocks for the preparation of MOF membrane due to their 2D morphology. Accordingly, various embodiments refer in a further aspect to a metal-organic framework membrane and a method of preparing a metal-organic framework membrane.

The method comprises preparing one or more metal-organic framework nanosheets according to the method of the first aspect or providing one or more metal-organic framework nanosheets according to the third aspect, dispersing the one or more metal-organic framework nanosheets in an aqueous solution to form a mixture, and filtering the mixture through a membrane, such as a nitrocellulose membrane filter or any other porous material, such that the one or more metal-organic framework nanosheets is deposited on the membrane to obtain the metal-organic framework membrane. The method of preparing a metal-organic framework membrane may further comprise drying the metal-organic framework membrane at room temperature.

Advantageously, by removing the membrane from the metal-organic framework membrane, a freestanding metal-organic framework membrane may be obtained. The metal-organic framework membrane may comprise a plurality of the metal-organic framework nanosheets disclosed herein, and which may be present in a layered structure due to stacking of the metal-organic framework nanosheets.

By virtue of the MOF nanosheets being porous, the resultant MOF membrane comprising a plurality of the metal-organic framework nanosheets according to embodiments disclosed herein may also be porous. The MOF membrane has been successfully used for separation of dyes with different sizes according to embodiments disclosed herein. The porosity of the MOF membrane allows separation of particles such as dye particles of different sizes, as particles which have a size that is larger than the pore size of the MOF membrane may not pass through the membrane and is retained on the MOF membrane.

Various embodiments thus refer in a further aspect to use of a metal-organic framework membrane disclosed herein or prepared by a method of preparing a metal-organic framework membrane disclosed herein in separation, preferably for separating organic dyes of different sizes. By controlling pore size of the MOF nanosheets formed, by for example, using ligands of different sizes, pore size of the resultant MOF membrane may in turn be controlled. This allows designing and/or tailoring of the MOF membrane according to requirements of specific applications. Details of how the separation may be carried out are provided in the examples. Advantageously, the MOF membrane may be recycled and the recycle of the MOF membrane is quite convenient, due to their good mechanical property and robust structure, which is important for their real application in separation.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

According to various embodiments disclosed herein, a facile surfactant-assisted bottom-up synthetic method to control growth kinetics of MOF crystals was developed. A series of freestanding ultrathin 2D MOF nanosheets with thickness of sub-10 nm were successfully prepared, including but not limited to M-TCPP (TCPP=tetrakis(4-carboxyphenyl)porphyrin, M=Zn, Cu, Cd, Co or Al) nanosheets, M-TCPP(M′) (M=Zn, Cu, Cd, Co and Al; M′=Fe, Co, Ni and Mn) nanosheets, M-BDC (BDC=terephthalic acid, M=Cu, Al) and M-BDC-NH₂ (BDC-NH₂₌₂-aminoterephthalic acid, M=Cu, Al) nanosheets.

Different with previously reported methods for the synthesis of bulk MOF crystals, by using the developed surfactant-assisted method disclosed herein, ultrathin 2D MOF nanosheets may be obtained directly. Importantly, due to the coverage of surfactants on their surface, the as-synthesized 2D MOF nanosheets were stable in solution without restacking.

Although top-down methods, such as exfoliation of the bulk MOF materials, have been used to prepare MOF nanosheets, yield of the exfoliation method is less than 15%, and restacking of the detached nanosheets may take place during the handling process. The bottom-up method in the form of direct synthesis of freestanding MOF nanosheets is promising but remains a challenge as it is difficult to control the growth kinetic of MOF crystals in solution phase.

The as-synthesized ultrathin 2D MOF nanosheets may be used directly or after modification for a number of applications such as sensing and separation.

For the first time, a bottom-up method in the form of a surfactant-assisted synthetic method to produce uniform ultrathin 2D MOF nanosheets with thickness of sub-10 nm is reported. For example, by using the developed surfactant-assisted method disclosed herein, 2D Zn-TCPP nanosheets may be obtained. In this kind of 2D MOF nanosheets, one TCPP ligand is linked by four Zn paddlewheel metal nodes, such as Zn₂(COO)₄, to form a 2D layered sheet. The layered sheets are further stacked in an AB packing pattern, forming 2D MOF structure with space group of I4/mmm.

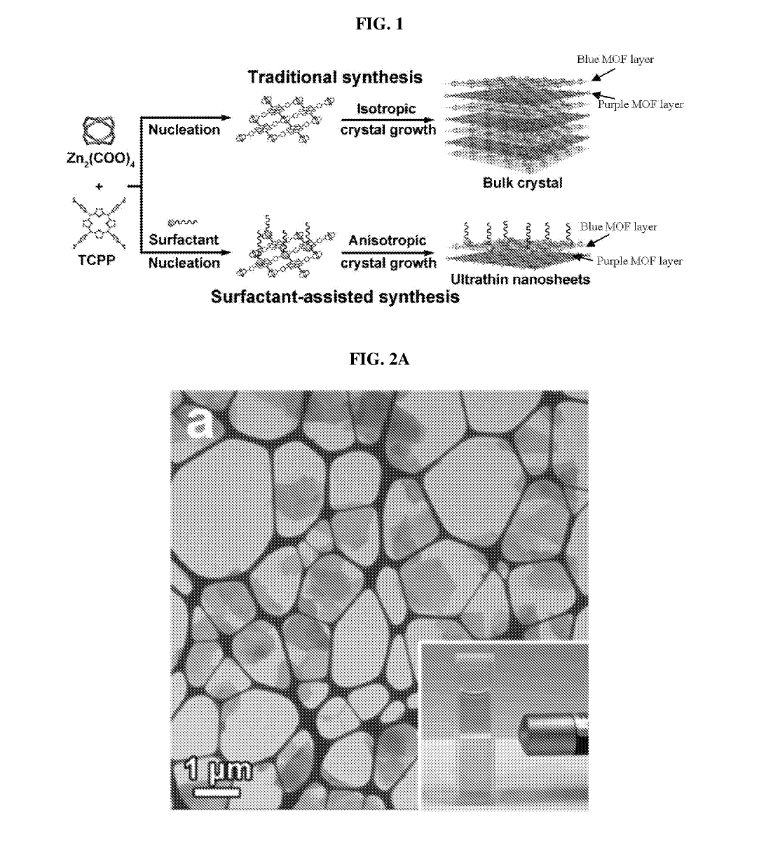

As disclosed herein, growth of MOFs in one dimension may be restricted using surfactants to form 2D MOF nanosheets. In experiments disclosed herein, polyvinylpyrrolidone (PVP) was used as surfactant during the preparation of Zn-TCPP. As shown in FIG. 1, by using the proposed surfactant-assisted synthetic method, the surfactant molecules can selectively attach on surface of MOFs, which play a key role for the controlled growth of MOF crystals, leading to anisotropic growth of MOFs and formation of ultrathin MOF nanosheets. In contrast thereto, by using traditional synthetic methods without surfactants (FIG. 1), there is isotropic growth of MOFs which results in MOFs in the form of bulk crystals.

Further details of various embodiments of the invention are listed as follow:

A surfactant-assisted synthetic method to prepare a series of freestanding ultrathin 2D MOF nanosheets with thickness of sub-10 nm is reported for the first time. These 2D MOF nanosheets include, but are not limited to M-TCPP (M=Zn, Cu, Cd, Co or Al) nanosheets, M-BDC (M=Cu, Al), and M-BDC-NH₂ (M=Cu, Al) nanosheets.

Surfactants were used for the direct bottom-up synthesis of freestanding ultrathin 2D MOF nanosheets. Surfactants which may be used include, but not limited to, polyvinylpyrrolidone (PVP) and hexadecyltrimethylammonium bromide (CTAB).

Hetero-metal freestanding ultrathin M-TCPP(M′) (where M includes but are not limited to Zn, Cu, Cd, Co and Al; and M′ includes, but are not limited to Fe, Co, Ni and Mn) nanosheets may also be produced by using metalated TCPP (metalation of TCCP) such as, but not limited to, TCPP(Fe), TCPP(Co), TCPP(Ni) and TCPP(Mn) as ligand instead of TCPP.

MOF membrane using 2D MOF nanosheets as building blocks may also be prepared, where freestanding MOF membrane may be obtained after removing of substrate.

Due to the easy and controlled synthesis process, the synthesis method disclosed herein is able to provide large quantity of target MOF nanosheets with desired composition, which is important for practical application.

The obtained ultrathin 2D MOF nanosheets may be directly used in or further modified for various applications, including but not limited to sensing and separation.

Usage of 2D MOF nanosheets in the applications of sensing and preparation of MOF membrane using 2D MOF nanosheets as building blocks, and their application in separation are presented for the first time herein.

The method in this disclosure offers great opportunities for commercialization of the 2D MOF based sensing platforms for the DNA detection (detection limit: 20 pM). Preliminary results show that the MOF membrane may be easily reused for subsequent cycles after washing and drying due to their good mechanical property.

Various embodiments refer to a method of producing a two dimensional metal organic framework nanosheet product. The method comprising dissolving a solution of metal precursor and ligand mixed with a surfactant, sonicating/stirring the solution, heating the solution and holding it for a specific time, washing the resultant nanosheet product with ethanol, centrifuging at a certain rpm, and redispersing the nanosheet product in ethanol.

Various embodiments refer to a two dimensional metal organic framework (MOF) nanosheet, comprising a metal precursor and a ligand, wherein its thickness is sub-10 nm. Products include Zn-TCPP, Cu-TCPP, Cd-TCPP, Co-TCPP, Al-TCPP, M-TCPP (M′) where M=Zn, Cu, Cd, Co & Al and M′=Fe, Co, Ni & Mn and Cu-BDC, Al-BDC and Cu-BDC-NH₂, Al-BDC-NH₂.

Various embodiments refer to a membrane synthesized from nanosheets as herein discussed, wherein the lateral size is about 1 μm.

Various embodiments refer to a method of synthesizing a MOF membrane, wherein the 2D MOF nanosheets were redispersed in water with a concentration of 0.1 mg mL⁻¹. Then the MOF membranes were prepared by suction filtration using nitrocellulose membrane filter as support. After filtration, the MOF membranes were dried at room temperature.

Various embodiments refer to a method for separating organic dyes with different molecular sizes, comprising filtering the dyes with the membrane as herein discussed.

Various embodiments refer to a method for detecting DNA, comprising probing single strand DNA and double strand DNA with a nanosheet (preferably Cu-TCPP) as herein discussed configured to quench the fluorescence of single strand DNA. Detection limit is 20 pM. A multiplexed DNA bioassay for the simultaneous detection of 2 genes is also made possible.

Various embodiments refer to a method for detecting H₂O₂ using MOF nanosheet, preferably Co-TCPP (Fe). The method is also deployed for real time detection of H₂O₂.

### Example 1: Materials

Zinc nitrate hexahydrate (Zn(NO₃)₂.6H₂O, 98%), Copper nitrate trihydrate (Cu(NO₃)₂.3H₂O, 99%), Cobalt nitrate hexahydrate (Co(NO₃)₂.6H₂O, 98%), Cadmium nitrate trihydrate (Cd(NO₃)₂.4H₂O, 98%), Polyvinylpyrrolidone (PVP, average mol wt 40,000), N,N-Dimethylformamide (DMF, 99.8%) were purchased from Sigma-Aldrich. Tetrakis(4-carboxyphenyl)porphyrin (TCPP, 97%) was purchased from Tokyo Chemical Industry Co. Ltd. Ethanol was purchased from Merck. Pyrazine (99%) and Trifluoroacetic acid (CF₃COOH, 99%) were purchased from Alfa Aesar. All DNA strands were synthesized and purified by the Integrated DNA Technologies Pte Ltd. The sequences of used DNA are listed in TABLE 1.

TABLE 1
DNA sequences used for the fluorescence assay

| Name | Sequence (5-3)* |
|---|---|
| H5N1 (T1) | CATACTGAGAACTCAAGAGTCT |
| | (SEQ ID NO: 1) |
| Probe (P1) | AGACTCTTGAGTTCTCAGTATG- |
| | Texas Red (SEQ ID NO: 2) |
| Single-base mismatch | CATATTGAGAACTCAAGAGTCT |
| DNA (SM1) | (SEQ ID NO: 3) |
| H1N1 (T2) | CGACTACACTCTCGATGAAGAA |
| | (SEQ ID NO: 4) |
| Probe (P2) | TTCTTCATCGAGAGTGTAGTCG-TET |
| | (SEQ ID NO: 5) |
| Single-base mismatch | CGACTACACTCTGGATGAAGAA |
| DNA (SM2) | (SEQ ID NO: 6) |
| Random DNA (R) | TAGCTTATCAGACAGATGTTGA |
| | (SEQ ID NO: 7) |

Abbreviations:
TET: tetrafluororescein.

The deionized water was obtained from the Milli-Q System. All the materials were used as received without further purification.

### Example 2: Synthesis of 2D MOF Nanosheets

### Example 2.1 Synthesis of Zn-TCPP Nanosheets

Zn(NO₃)₂.6H₂O (4.5 mg, 0.015 mmol), pyrazine (0.8 mg, 0.01 mmol) and PVP (20.0 mg) in 12 mL of the mixture of DMF and ethanol (V:V=3:1) were dissolved in a 20 mL capped vial. Then TCPP (4.0 mg, 0.005 mmol) dissolved in 4 mL of the mixture of DMF and ethanol (V:V=3:1) were added in a dropwise manner under stirring. After that, the solution was sonicated for 10 min. The vial was heated to 80° C. and then kept the reaction for 16 h. The resulting purple nanosheets were washed twice with ethanol and collected by centrifuging at 8,000 r.p.m. for 10 min. Finally, the obtained Zn-TCPP nanosheets were redispersed in 10 mL of ethanol.

### Example 2.2: Synthesis of Cd-TCPP Nanosheets

Cd(NO₃)₂.4H₂O (4.6 mg, 0.015 mmol), pyrazine (0.8 mg, 0.01 mmol) and PVP (20.0 mg) in 12 mL of the mixture of DMF and ethanol (V:V=3:1) were dissolved in a 20 mL capped vial. Then TCPP (4.0 mg, 0.005 mmol) dissolved in 4 mL of the mixture of DMF and ethanol (V:V=3:1) were added dropwisely under stirring. After that, the solution was sonicated for 10 min. The vial was heated to 80° C. and then kept the reaction for 24 h. The resulting dark green nanosheets were washed twice with ethanol and collected by centrifuging at 8,000 r.p.m. for 10 min. Finally, the obtained Cd-TCPP nanosheets were redispersed in 10 mL of ethanol.

### Example 2.3: Synthesis of Cu-TCPP Nanosheets

Cu(NO₃)₂.3H₂O (3.6 mg, 0.015 mmol), trifluoroacetic acid (1.0 M×10 μL) and PVP (10.0 mg) in 12 mL of the mixture of DMF and ethanol (V:V=3:1) were dissolved in a 20 mL capped vial. Then TCPP (4.0 mg, 0.005 mmol) dissolved in 4 mL of the mixture of DMF and ethanol (V:V=3:1) were added dropwisely under stirring. After that, the solution was sonicated for 10 min. The vial was heated to 80° C. and then kept the reaction for 3 h. The resulting red nanosheets were washed twice with ethanol and collected by centrifuging at 8,000 r.p.m. for 10 min. Finally, the obtained Cu-TCPP nanosheets were redispersed in 10 mL of ethanol.

### Example 2.4: Synthesis of Co-TCPP Nanosheets

Co(NO₃)₂.6H₂O (4.4 mg, 0.015 mmol), pyrazine (0.8 mg, 0.01 mmol) and PVP (20.0 mg) in 12 mL of the mixture of DMF and ethanol (V:V=3:1) were dissolved in a 20 mL capped vial. Then TCPP (4.0 mg, 0.005 mmol) dissolved in 4 mL of the mixture of DMF and ethanol (V:V=3:1) were added dropwisely under stirring. After that, the solution was sonicated for 10 min. The vial was heated to 80° C. and then kept the reaction for 24 h. The resulting red nanosheets were washed twice with ethanol and collected by centrifuging at 8,000 r.p.m. for 10 min. Finally, the obtained Co-TCPP nanosheets were redispersed in 10 mL of ethanol.

### Example 2.5: Synthesis of Zn-TCPP(Fe) Nanosheets

Zn(NO₃)₂.6H₂O (3.0 mg, 0.01 mmol), pyrazine (0.8 mg, 0.01 mmol) and PVP (20.0 mg) in 12 mL of the mixture of DMF and ethanol (V:V=3:1) were dissolved in a 20 mL capped vial. Then [tetrakis(4-carboxyphenyl)porphyrinato]-Fe(III) chloride, TCPP(Fe) (4.4 mg, 0.005 mmol) dissolved in 4 mL of the mixture of DMF and ethanol (V:V=3:1) were added dropwisely under stirring. After that, the solution was sonicated for 10 min. The vial was heated to 80° C. and then kept the reaction for 24 h. The resulting dark brown nanosheets were washed twice with ethanol and collected by centrifuging at 8,000 r.p.m. for 10 min. Finally, the obtained Zn-TCPP(Fe) nanosheets were redispersed in 10 mL of ethanol.

### Example 2.6: Synthesis of Cu-TCPP(Fe) Nanosheets

Cu(NO₃)₂.3H₂O (2.4 mg, 0.01 mmol), trifluoroacetic acid (1.0 M×10 μL) and PVP (20.0 mg) in 12 mL of the mixture of DMF and ethanol (V:V=3:1) were dissolved in a 20 mL capped vial. Subsequently, [tetrakis(4-carboxyphenyl)porphyrinato]-Fe(III) chloride, TCPP(Fe) (4.4 mg, 0.005 mmol) dissolved in 4 mL of the mixture of DMF and ethanol (V:V=3:1) were added dropwisely under stirring. After that, the solution was sonicated for 10 min. The vial was heated to 80° C. and the reaction was kept for 24 h. The resulting dark brown nanosheets were washed twice with ethanol and collected by centrifuging at 8,000 r.p.m. for 10 min. Finally, the obtained Cu-TCPP(Fe) nanosheets were redispersed in 10 mL of ethanol.

### Example 2.7: Synthesis of Co-TCPP(Fe) Nanosheets

Co(NO₃)₂.6H₂O (2.4 mg, 0.01 mmol), pyrazine (0.8 mg, 0.01 mmol) and PVP (20.0 mg) in 12 mL of the mixture of DMF and ethanol (V:V=3:1) were dissolved in a 20 mL capped vial. Subsequently, [tetrakis(4-carboxyphenyl)porphyrinato]-Fe(III) chloride, TCPP(Fe) (4.4 mg, 0.005 mmol) dissolved in 4 mL of the mixture of DMF and ethanol (V:V=3:1) were added in a dropwise manner under stirring. After that, the solution was sonicated for 10 min. The vial was heated to 80° C. and the reaction was kept for 24 h. The resulting dark brown nanosheets were washed twice with ethanol and collected by centrifuging at 8,000 r.p.m. for 10 min. Finally, the obtained Co-TCPP(Fe) nanosheets were redispersed in 10 mL of ethanol.

### Example 2.8: Synthesis of Cu-TCPP(Co) Nanosheets

Cu(NO₃)₂.3H₂O (2.4 mg, 0.01 mmol), trifluoroacetic acid (1.0 M×10 μL) and PVP (20.0 mg) in 12 mL of the mixture of DMF and ethanol (V:V=3:1) were dissolved in a 20 mL capped vial. Subsequently, [tetrakis(4-carboxyphenyl)porphyrinato]-Co(III) chloride, TCPP(Co) (4.4 mg, 0.005 mmol) dissolved in 4 mL of the mixture of DMF and ethanol (V:V=3:1) were added in a dropwise manner under stirring. After that, the solution was sonicated for 10 min. The vial was heated to 80° C. and the reaction was kept for 24 h. The resulting dark red nanosheets were washed twice with ethanol and collected by centrifuging at 8,000 r.p.m. for 10 min. Finally, the obtained Cu-TCPP(Co) nanosheets were redispersed in 10 mL of ethanol.

### Example 2.9: Synthesis of Cu-TCPP(Mn) Nanosheets

Cu(NO₃)₂.3H₂O (2.4 mg, 0.01 mmol), trifluoroacetic acid (1.0 M×10 μL) and PVP (20.0 mg) in 12 mL of the mixture of DMF and ethanol (V:V=3:1) were dissolved in a 20 mL capped vial. Subsequently, [tetrakis(4-carboxyphenyl)porphyrinato]-Mn(III) chloride, TCPP(Mn) (4.4 mg, 0.005 mmol) dissolved in 4 mL of the mixture of DMF and ethanol (V:V=3:1) were added in a dropwise manner under stirring. After that, the solution was sonicated for 10 min. The vial was heated to 80° C. and the reaction was kept for 24 h. The resulting dark brown nanosheets were washed twice with ethanol and collected by centrifuging at 8,000 r.p.m. for 10 min. Finally, the obtained Cu-TCPP(Mn) nanosheets were redispersed in 10 mL of ethanol.

### Example 2.10: Synthesis of Cu-TCPP(Ni) Nanosheets

Cu(NO₃)₂.3H₂O (2.4 mg, 0.01 mmol), trifluoroacetic acid (1.0 M×10 μL) and PVP (20.0 mg) in 12 mL of the mixture of DMF and ethanol (V:V=3:1) were dissolved in a 20 mL capped vial. Subsequently, [tetrakis(4-carboxyphenyl)porphyrinato]-Ni(II), TCPP(Ni) (4.4 mg, 0.005 mmol) dissolved in 4 mL of the mixture of DMF and ethanol (V:V=3:1) were added in a dropwise manner under stirring. After that, the solution was sonicated for 10 min. The vial was heated to 80° C. and the reaction was kept for 24 h. The resulting dark red nanosheets were washed twice with ethanol and collected by centrifuging at 8,000 r.p.m. for 10 min. Finally, the obtained Cu-TCPP(Ni) nanosheets were redispersed in 10 mL of ethanol.

### Example 2.11: Synthesis of Al-TCPP Nanosheets

AlCl₃.6H₂O (4.8 mg, 0.02 mmol) dissolved in 12 mL of water in a 23 mL autoclave liner. Subsequently, TCPP (8.0 mg, 0.01 mmol) and CTAB (20.0 mg) dissolved in 2 mL of ethanol were added in a dropwise manner under stirring. After that, the solution was stirred for 10 min. The autoclave was heated to 160° C. and the reaction was kept for 8 h. The resulting purple nanosheets were washed twice with ethanol and collected by centrifuging at 8,000 r.p.m. for 10 min. Finally, the obtained Al-TCPP nanosheets were redispersed in 10 mL of ethanol.

### Example 2.12: Synthesis of Al-TCPP(M′, M′=Fe, Co, Mn and Ni) Nanosheets

AlCl₃.6H₂O (4.8 mg, 0.02 mmol) was dissolved in 12 mL of water in a 23 mL autoclave liner. Subsequently, TCPP(M′) (8.8 mg, 0.01 mmol) and CTAB (20.0 mg) dissolved in 2 mL of ethanol were added in a dropwise manner under stirring. After that, the solution was stirred for 10 min. The autoclave was heated to 160° C. and the reaction was kept for 8 h. The resulting purple nanosheets were washed twice with ethanol and collected by centrifuging at 8,000 r.p.m. for 10 min. Finally, the obtained Al-TCPP nanosheets were redispersed in 10 mL of ethanol.

### Example 2.13: Synthesis of Cu-BDC Nanosheets

Cu(NO₃)₂.3H₂O (12 mg, 0.04 mmol), pyridine (40 μL, 0.5 mmol) and PVP (10.0 mg) in 6 mL of the mixture of DMF and ethanol (V:V=3:1) were dissolved in a 20 mL capped vial. Subsequently, BDC (10.0 mg, 0.06 mmol) dissolved in 4 mL of the mixture of DMF and ethanol (V:V=3:1) were added dropwisely under stirring. After that, the solution was sonicated for 10 min. The vial was heated to 60° C. and the reaction was kept for 10 h. The resulting blue nanosheets were washed twice with ethanol and collected by centrifuging at 8,000 r.p.m. for 10 min. Finally, the obtained Cu-BDC nanosheets were redispersed in 10 mL of ethanol.

### Example 2.14: Synthesis of Cu-BDC-NH₂ Nanosheets

Cu(NO₃)₂.3H₂O (12 mg, 0.04 mmol), pyridine (40 μL, 0.5 mmol) and PVP (10.0 mg) in 6 mL of the mixture of DMF and ethanol (V:V=3:1) were dissolved in a 20 mL capped vial. Subsequently, BDC-NH₂ (11.0 mg, 0.06 mmol) dissolved in 4 mL of the mixture of DMF and ethanol (V:V=3:1) were added dropwisely under stirring. After that, the solution was sonicated for 10 min. The vial was heated to 60° C. and the reaction was kept for 10 h. The resulting blue nanosheets were washed twice with ethanol and collected by centrifuging at 8,000 r.p.m. for 10 min. Finally, the obtained Cu-BDC-NH₂ nanosheets were redispersed in 10 mL of ethanol.

### Example 2.15: Synthesis of Al-BDC Nanosheets

AlCl₃.6H₂O (12.0 mg, 0.05 mmol) was dissolved in 5 mL of water in a 23 mL autoclave liner. Subsequently, BDC (8.3 mg, 0.05 mmol) and CTAB (10.0 mg) dissolved in 1 mL of ethanol were added in a dropwise manner under stirring. After that, the solution was stirred for 10 min. The autoclave was heated to 160° C. and the reaction was kept for 20 h. The resulting white nanosheets were washed twice with ethanol and collected by centrifuging at 8,000 r.p.m. for 10 min. Finally, the obtained Al-BDC nanosheets were redispersed in 10 mL of ethanol.

### Example 2.16: Synthesis of Al-BDC-NH₂ Nanosheets

AlCl₃.6H₂O (12.0 mg, 0.05 mmol) was dissolved in 5 mL of water in a 23 mL autoclave liner. Subsequently, BDC (9.0 mg, 0.05 mmol) and CTAB (10.0 mg) dissolved in 1 mL of ethanol were added in a dropwise manner under stirring. After that, the solution was stirred for 10 min. The autoclave was heated to 160° C. and the reaction was kept for 20 h. The resulting yellow nanosheets were washed twice with ethanol and collected by centrifuging at 8,000 r.p.m. for 10 min. Finally, the obtained Al-BDC-NH₂ nanosheets were redispersed in 10 mL of ethanol.

### Example 2.17: Synthesis of MOF Membrane

The as-prepared ultrathin 2D MOF nanosheets were redispersed in water with a concentration of 0.1 mg mL⁻¹. The MOF membranes were then prepared by suction filtration using nitrocellulose membrane filter as support. After filtration, the MOF membrane were dried at room temperature.

### Example 2.18: Synthesis of MIL-69/Al-TCPP Heterostructure

AlCl₃.6H₂O (24 mg, 0.1 mmol) was dissolved in 10 mL of water in a 23 mL autoclave liner. Subsequently, 2,6-naphthalenedicarboxylic acid (21.6 mg, 0.01 mmol), and pyridine (50 μL) dissolved in 2 mL of ethanol were added in a dropwise manner under stirring. Pyridine was used to facilitate the dissolution of 2,6-naphthalenedicarboxylic acid ligand. After that, the solution was stirred for 10 min. The autoclave was heated to 160° C. and the reaction was kept for 20 h. The resulting white products were termed “MIL-69” and were washed twice with water, and collected by centrifuging at 8,000 r.p.m. for 10 min. Finally, the obtained MIL-69 were redispersed in 10 mL of water.

AlCl₃.6H₂O (4.8 mg, 0.02 mmol) was added to the abovementioned MIL-69 solution in a 23 mL autoclave liner. TCPP (8.0 mg, 0.01 mmol) and CTAB (10.0 mg) dissolved in 2 mL of ethanol were then added in a dropwise manner under stirring. After that, the solution was stirred for 10 min. The autoclave was heated to 160° C. and the reaction was kept for 10 h. The resulting purple products were washed twice with ethanol and collected by centrifuging at 8,000 r.p.m. for 10 min. Finally, the obtained MIL-69/Al-TCPP heterostructure were redispersed in 10 mL of ethanol.

### Example 2.19: Synthesis of MIL-68/in-TCPP Heterostructure

In(NO₃)₃ (43 mg, 0.1 mmol) was dissolved in 2 mL DMF in a 10 mL glass vial. Subsequently, BDC (16.6 mg, 0.1 mmol), and pyridine (10 uL) dissolved in 2 mL of DMF were added in a dropwise manner under stirring. Pyridine was used to facilitate the dissolution of BDC ligand. After that, the solution was heated to 80° C. in an oil bath and the reaction was kept for 2 h. The resulting white products were termed “MIL-68” and were washed twice with DMF, and collected by centrifuging at 8,000 r.p.m. for 10 min. Finally, the obtained MIL-68 were redispersed in 5 mL of DMF.

In(NO₃)₃ (4.8 mg, 0.02 mmol) was added to the abovementioned MIL-68 solution in a 10 mL glass vial. Then TCPP (4.0 mg, 0.005 mmol) dissolved in 2 mL of DMF were added dropwisely under stirring. After that, the solution was stirred for 10 min. The solution was heated to 140° C. and then kept the reaction for 10 h. The resulting purple products were washed twice with ethanol and collected by centrifuging at 8,000 r.p.m. for 10 min. Finally, the obtained MIL-68/In-TCPP heterostructure were redispersed in 5 mL of DMF.

### Example 2.20: Synthesis of MOF-525/Zr-BTB Heterostructure

ZrOCl₂.8H₂O (11 mg, 0.034 mmol) was dissolved in 3 mL DMF and acetic acid (0.7 mL) in a 10 mL glass vial. TCPP (20 mg, 0.025 mmol) dissolved in 3 mL of DMF were then added in a dropwise manner under stirring. After that, the solution was heated to 90° C. in an oil bathe and the reaction was kept for 15 h. The resulting purple products were termed “MOF-525” and were washed twice with DMF, and collected by centrifuging at 8,000 r.p.m. for 10 min. Finally, the obtained MOF-525 were redispersed in 5 mL of DMF.

ZrOCl₂.8H₂O (11 mg, 0.034 mmol), biphenyl-4-carboxylic acid (200 mg) were added to the abovementioned MOF-525 solution in a 10 mL glass vial. After heating at 90° C. for 1 h, 1,3,5-Tris(4-carboxyphenyl)benzene (BTB) (10.0 mg, 0.023 mmol) was added, and the solution was further heated to 120° C. and reaction was kept for 6 h. The resulting purple products were washed twice with ethanol and collected by centrifuging at 8,000 r.p.m. for 10 min. Finally, the obtained MIL-68/In-TCPP heterostructure were redispersed in 5 mL of DMF.

### Example 2.21: Synthesis of Au/Cu-TCPP(Fe) Hybrid Nanomaterial

100 μL of HAuCl₄ (15 mM) was added into 5 mL of Cu-TCPP(Fe) (0.1 mg mL⁻¹) nanosheet aqueous solution. The mixture was stirred for 1 min at room temperature, followed by the addition of 25 μL of NaBH₄ aqueous solution (0.1 M). Then the mixture was rinsed with water twice and centrifuged at 10000 r.p.m for 10 min. Finally, the obtained Au/Cu-TCPP(Fe) nanosheets were redispersed in water.

### Example 3: Fluorescent DNA Assays

In a typical hybridization experiment, prior to the addition of 2D Cu-TCPP nanosheet solution (50 μL, 1.4 mg mL⁻¹), 5 μL of probe P1 (1 μM) were hybridized with 10 μL of the target DNA (T1) (0-4 μM) in 1935 μL of phosphate buffer saline (0.01 M, pH 7.4) for 30 min at room temperature. After incubation for 5 min, fluorescence measurements were performed to monitor the hybridization process with the final concentration of T1 (0-20 nM). The excitation and emission wavelengths were fixed at 588 and 609 nm.

For the comparison study, 5 μL of probe P1 (1 μM) were hybridized with 10 μL of the target DNA (T1) (4 μM) in 1935 μL of phosphate buffer saline (0.01 M, pH 7.4) for 30 min at room temperature. Then, 50 μL of bulk Cu-TCPP MOFs (1.4 mg mL⁻¹), or Zn-TCPP(Fe) nanosheets (1.4 mg mL⁻¹), or bulk Zn-TCPP(Fe) MOFs (1.4 mg mL⁻¹), or Co-TCPP nanosheets (1.4 mg mL⁻¹), or bulk Co-TCPP MOFs (1.4 mg mL⁻¹) solution was added into the aforementioned mixture. After incubation for 5 min, fluorescence measurements were performed to monitor the hybridization process with the final concentration of T1 (20 nM).

In the control experiment, 5 μL of probe P1 (1 μM) were hybridized with 10 μL of the target DNA (T1) (4 μM) in 1935 μL of phosphate buffer saline (0.01 M, pH 7.4) for 30 min at room temperature. Then, 50 μL of metal ions (4.2 mM Cu²⁺, or 2.8 mM Zn²⁺ and 1.4 mM Fe³⁺, or 4.2 mM Co²⁺), or TCPP ligand (1.4 mM), or the mixture solution of metal ions (4.2 mM Cu²⁺, or 2.8 mM Zn²⁺ and 1.4 mM Fe³⁺, or 4.2 mM Co²⁺) and TCPP ligand (1.4 mM), or PVP with a serial of concentrations (0-1.4 mg mL⁻¹) was added into the aforementioned mixtures. Note that the concentrations of metal ions and TCPP ligand used in the control experiment were same with the concentrations of corresponding components (metal ions and TCPP ligand) in the 2D MOF nanosheets. After incubation for 5 min, fluorescence measurements were performed to monitor the hybridization process with the final concentration of T1 (20 nM).

In a multiplexed DNA assay, 5 μL of P1 (1 μM) and 5 μL of P2 (1 μM) were mixed with the phosphate buffer saline (10 μL, 0.01 M, pH 7.4, as control), or T1 (10 μL, 4 μM), or T2 (10 μL, 4 μM), or the mixture of T1 (5 μL, 8 μM) and T2 (5 μL, 8 μM), respectively, in 1930 μL of phosphate buffer saline (0.01 M, pH 7.4) for 30 min at room temperature. Then, 50 μL of 2D Cu-TCPP solution (1.4 mg mL⁻¹) were added into the aforementioned mixtures. After 5 min incubation, the fluorescence measurements were carried out with the final concentrations of T1 (20 nM) or T2 (20 nM). The excitation/emission wavelengths were fixed at 588/609 nm for P1, and 522/539 nm for P2, respectively.

In order to determine the concentration of 2D MOF nanosheets in water solution, after drying a certain amount of solution at 100° C., the weight of dry MOF nanosheets was measured by the balance (Mettler Toledo MT5).

For ssDNA, the quenching efficiency was defined as (1−F_{P1}/F₀)×100%, where F_{P1} and F₀ are the fluorescence intensity of ssDNA (P1) at 609 nm in the presence and absence of the quenchers, respectively. For dsDNA, the quenching efficiency was defined as (1−F_{P1/T1}/F₀)×100%, where F_{P1/T1} and F₀ are the fluorescence intensity of ds DNA (P1/T1) at 609 nm in the presence and absence of the quechers, respectively.

### Example 4: Electrochemical Biosensing

Before surface modification, glassy carbon electrode (GCE, 3 mm diameter) was polished with successively finer grade aqueous alumina slurries (grain size 5-0.5 μm) on a polishing cloth. 2D Co-TCPP(Fe) nanosheets was assembled on the GCE as follows: the 2D Co-TCPP(Fe) nanosheets were dispersed in ethanol to form a dark green colloidal suspension (1.0 mg mL⁻¹); second, the suspension was placed dropwisely onto the surface of water to form a Co-TCPP(Fe) thin film; finally, the thin film was transferred to GCE by “stamping.” Repetition of the above process led to the fabrication of the Co-TCPP(Fe) thin film on GCE. The HRP modified GCE (HRP/GCE) were prepared by drop-casting 10 μL of HRP dispersion on the GCE, respectively.

All the electrochemical measurements were carried out on a CHI 760D electrochemical workstation (CH Instruments, USA). The electrochemical cell was assembled with a conventional three electrode system: a saturated Ag/AgCl reference electrode, a Pt wire auxiliary electrode, and the prepared working electrode. Amperometric studies were carried out under stirred conditions. The sample solutions were purged with nitrogen for at least 30 min to remove oxygen prior to starting a series of experiments.

Human breast cancer cells (MDA MB 231) were obtained from ATCC (USA). The cells were maintained in a culture medium comprising Dulbecco's modified minimum essential medium (37° C., 5% CO₂) and subcultured every 3 days. After centrifuging, the cells were planted at a density of 5×10⁴ cell mL⁻¹ in a 24-well microplate. Cells at a confluency of 80% were used for the electrochemical experiments.

### Example 5: Separation of Dyes

As-prepared MOF membrane were fixed enclosed in a suction filter housing. 20 PPM aqueous solutions of organic dyes were added. The filtrate was collected and analyzed by UV-visible spectroscopy to determine dye content.

### Example 6: Characterization

Samples for TEM, SEM and AFM characterization were prepared by dropping the ethanolic suspensions of 2D MOF nanosheets onto holey carbon-coated carbon support copper grids, Si/SiO₂, and piranha-cleaned Si/SiO₂ respectively.

TEM was operated at an acceleration voltage of 200 kV (JEOL-2010UHR).

SEM images were obtained using a field emission scanning electron microscope (FE-SEM, JSM-7600F).

Bright-field STEM images were obtained using FE-SEM with a transmission electron detector (TED) operated at an accelerating voltage of 30 kV.

AFM (Cypher, Asylum Research) was used to characterize the 2D MOF nanosheets in tapping mode in air.

Powder X-ray diffraction (XRD) patterns were recorded with a Shimadzu XRD-6000 powder X-ray diffractometer, using Cu Kα radiation (λ=1.5406 Å).

The measurement of Brunauer-Emmett-Teller (BET) surface area and pore size was performed in a Micromeritics ASAP 2020 adsorption apparatus at 77 K and pressure up to 1 bar.

Fourier transform infrared (FTIR) spectra were collected on a Perkin Elmer FT-IR Spectrum GX in the spectral range of 400-4000 cm⁻¹ using the KBr disk method.

UV-vis absorption spectra were recorded at room temperature (UV-2700, Shimadzu) with QS-grade quartz cuvettes (111-QS, Hellma Analytics).

Fluorescence spectra were recorded with a Shimadzu fluorophotometer (RF-5301PC).

### Example 7: Results and Discussion

The obtained 2D Zn-TCPP nanosheets were characterized by scanning transmission electron microscopy (STEM), transmission electron microscopy (TEM), powder X-ray diffraction (XRD) and atomic force microscopy (AFM).

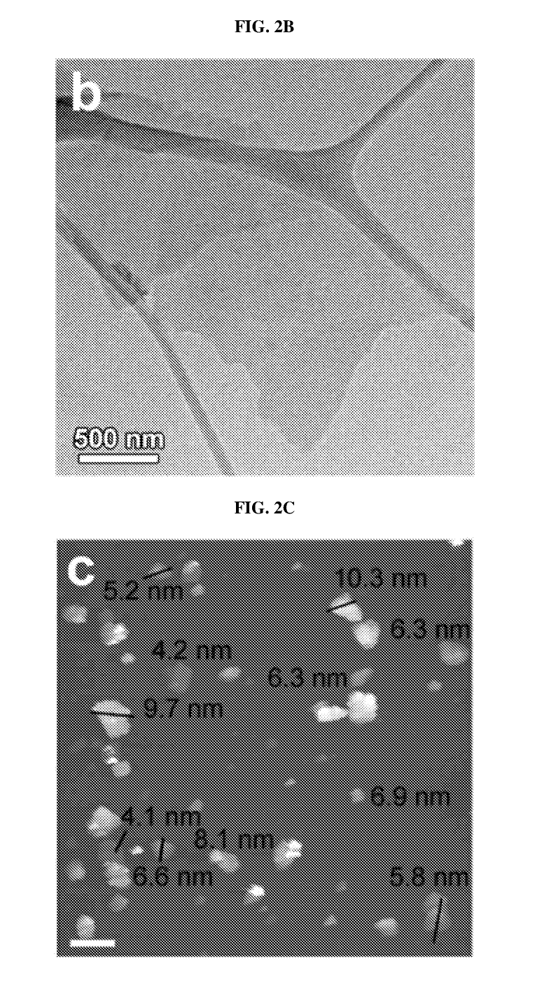

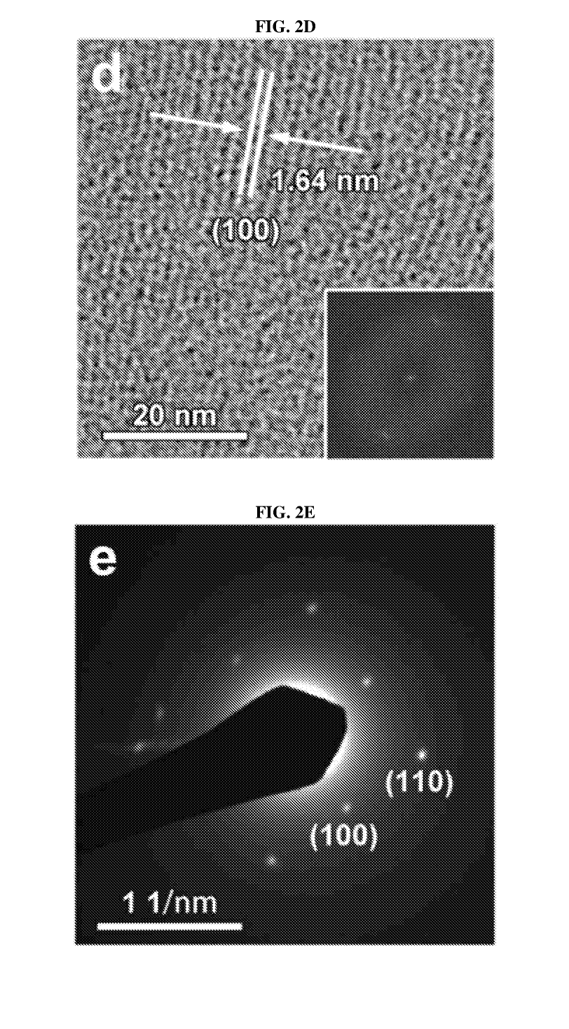

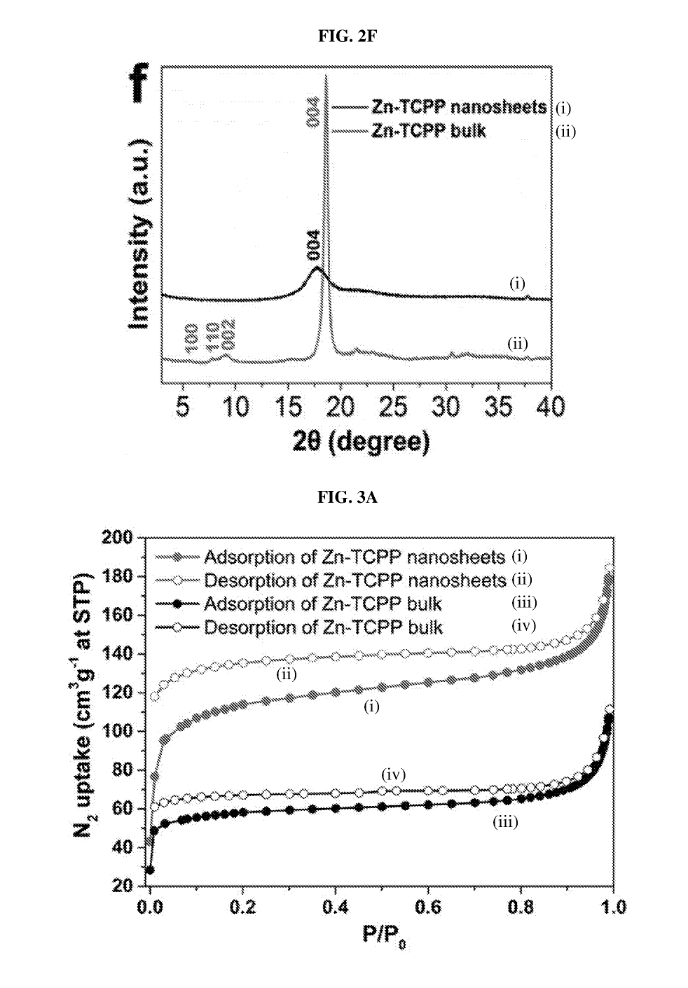

The bright-field STEM image clearly reveals the Zn-TCPP nanosheets with lateral size of 1.2±0.4 μm (FIG. 2A). The Tyndall effect observed in the ethanolic solution of Zn-TCPP nanosheets (inset in FIG. 2A) confirmed their colloidal structure.

The low contrast of Zn-TCPP nanosheets in TEM image proves their ultrathin nature (FIG. 2B). By using AFM, the thickness of Zn-TCPP nanosheets (7.6±2.6 nm) was measured (FIG. 2C). Since the theoretical interlayer distance of Zn-TCPP is 0.93 nm based on the X-ray structure model, the layer number of obtained Zn-TCPP nanosheets was about 8±3.

In order to confirm the crystal structure of Zn-TCPP, XRD characterization was performed. The bulk Zn-TCPP MOFs showed four typical peaks (FIG. 2F, red curve), which may be ascribed to the tetragonal structure of Zn-TCPP. Due to the lack of long-range ordered structure of ultrathin nanosheets, only a broad peak corresponding to the (004) plane was observed, indicating the crystal nature of Zn-TCPP nanosheets (FIG. 2F, black curve). As known, most of MOF materials are unstable under the irradiation of electron beam during the characterization by high-resolution TEM (HRTEM), resulting in the rapid destroy of their structure. So far, the lattice fringes may only be observed in a few stable MOFs, such as MIL-101, MOF-74 and UIO-MOF.

The inventors were able to visualize the lattice fringes of the Zn-TCPP nanosheet, indicating its excellent stability. The HRTEM image of Zn-TCPP nanosheet showed a lattice fringe with interplanar distance of 1.64 nm (FIG. 2D), which may be ascribed to the (100) plane of Zn-TCPP crystal since it is consistent with the value of 1.67 nm based on the X-ray structure model. The corresponding fast Fourier transform (FFT) analysis of Zn-TCPP nanosheet showed a 4-fold symmetry (inset in FIG. 2C), indicating its tetragonal crystal structure. The selected-area electron diffraction (SAED) pattern provided the diffraction spots (FIG. 2E), which may be attributed to the (110) and (100) planes of Zn-TCPP nanosheets.

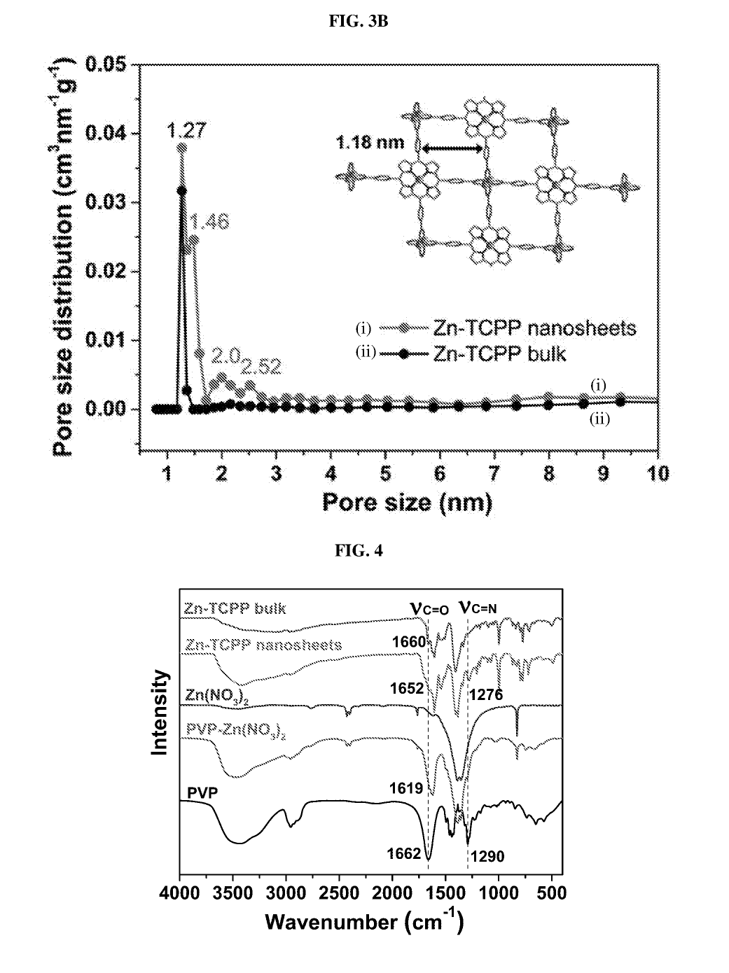

The porosity of Zn-TCPP nanosheets and bulk Zn-TCPP MOFs were examined by N₂ adsorption experiments (FIGS. 3A and 3B). FIG. 3A confirms that they showed similar approximate type I Langmuir isotherms. The Brunauer-Emmett-Teller (BET) surface area of bulk Zn-TCPP MOFs is 197 m²g⁻¹, while the BET surface area of Zn-TCPP nanosheet significantly increased to 391 m²g⁻¹.

In FIG. 3B, the pore size distribution data indicate that both of Zn-TCPP nanosheets and bulk Zn-TCPP MOFs have the same micropores of 1.27 nm, which is consistent with the value of 1.18 nm based on crystallographic data. However, the Zn-TCPP nanosheets also have other pores with size of 1.46, 2.0 and 2.52 nm, which could be attributed to the slit-like pores formed by aggregation of Zn-TCPP nanosheets during freeze drying. The similar N₂ adsorption isotherms and pore size indicate that the surfactant-assisted method disclosed herein has little effect on the pore structure of Zn-TCPP nanosheets.

In order to understand the role of surfactant, PVP, in the synthesis of Zn-TCPP nanosheets, Fourier transform infrared (FTIR) spectroscopy were performed. As shown in FIG. 4, the stretching vibration of ν_{C═O} in PVP at 1662 cm⁻¹ shifted to 1619 cm⁻¹ after mixing with Zn(NO₃)₂, indicating the strong interaction of C═O group in PVP with Zn²⁺ ions. Therefore, PVP could attach on the surface of MOF after nucleation, leading to the anisotropic growth of MOFs and finally form ultrathin Zn-TCPP nanosheets.

The aforementioned results demonstrated that the surfactant-assisted synthetic method disclosed herein has been successfully used to prepare ultrathin 2D Zn-TCPP nanosheets. More importantly, the method may be used to prepare various kinds of 2D MOF nano sheets.

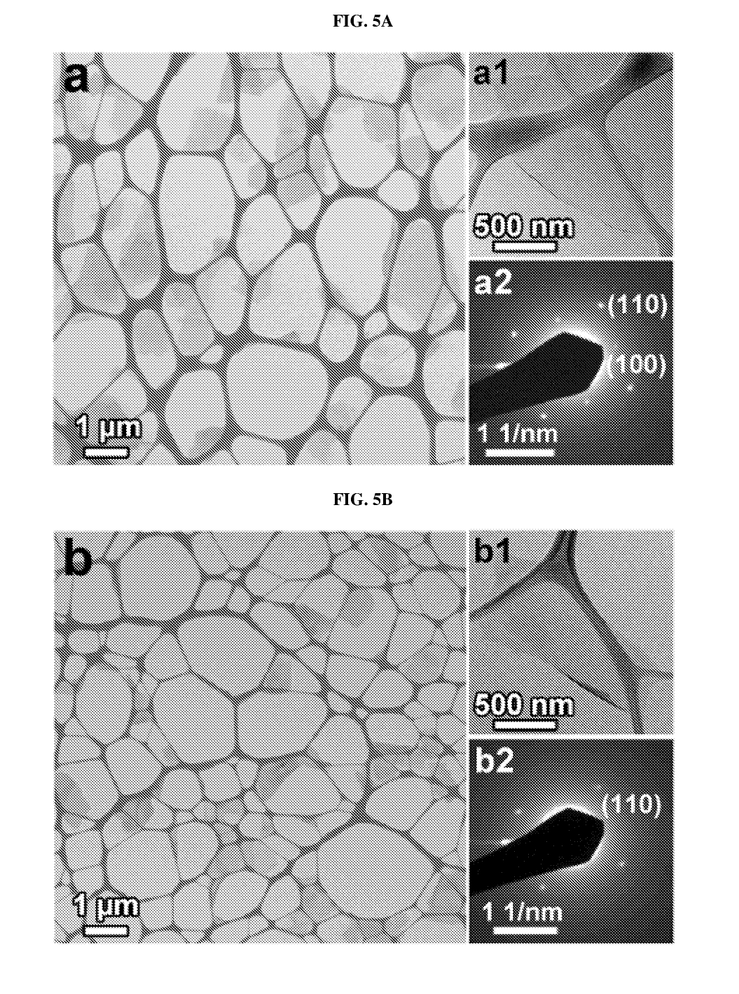

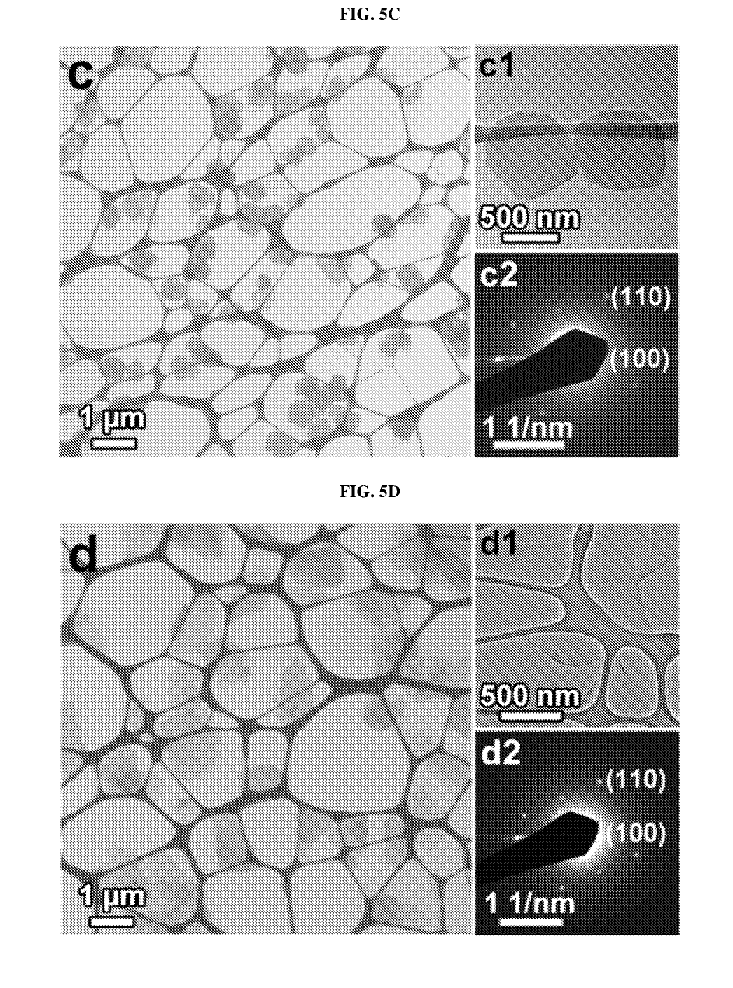

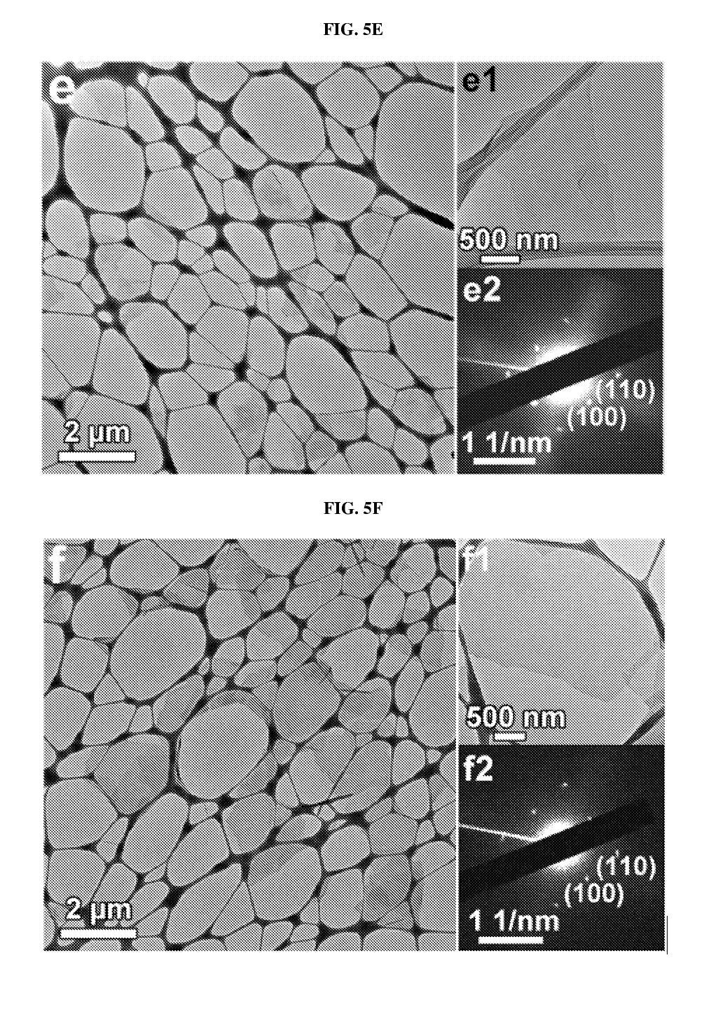

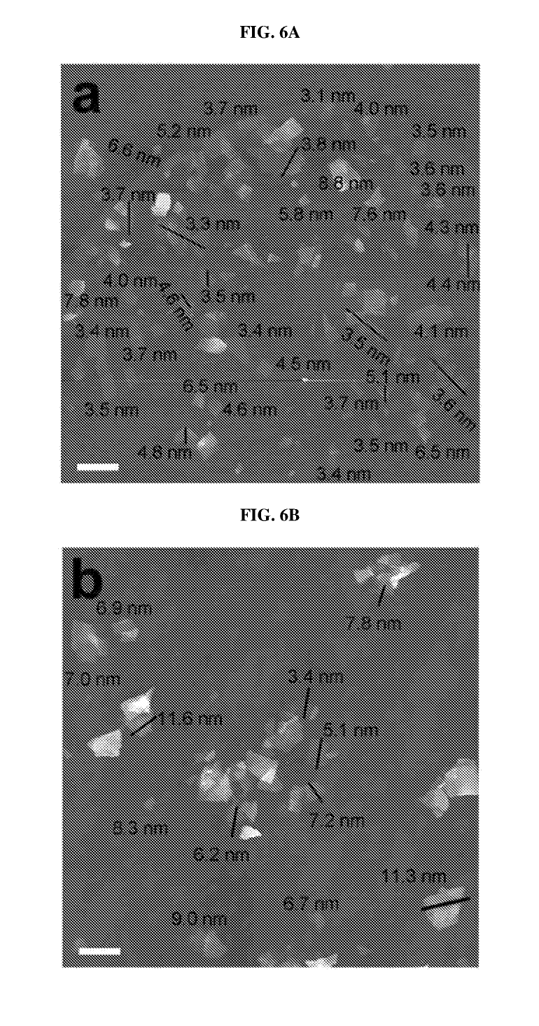

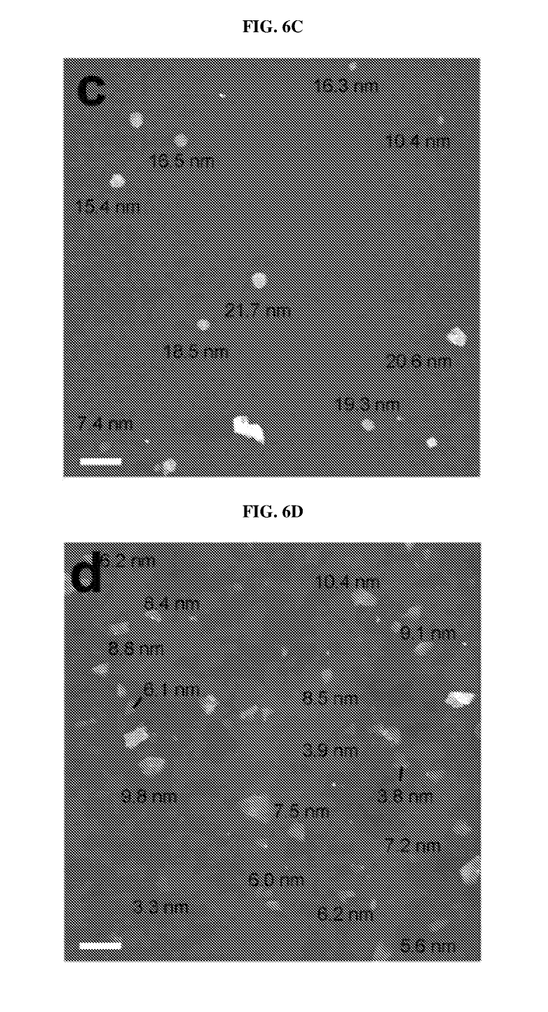

For example, when Cu(NO₃)₂ was used to replace Zn(NO₃)₂ as the metal precursor, well-dispersed Cu-TCPP nanosheets with lateral size of 1.0±0.2 μm were obtained (FIG. 5A). AFM image reveals that the thickness of Cu-TCPP nanosheet is 4.5±1.2 nm (FIG. 6A).

Similarly, the Cd-TCPP nanosheets with lateral size of 1.2±0.3 μm and thickness of 8.7±2.7 nm were also prepared (FIG. 5B and FIG. 6B). In the case of Co-TCPP, Co-TCPP with lateral size of 0.7±0.1 μm and thickness of 18.8±6.4 nm was prepared (FIG. 5C and FIG. 6C).

As known, many natural compounds contain the porphyrin units. For example, heme, an iron-porphyrin derivative, is the cofactor of many enzymes or proteins. In this work, a heme-like ligand, [tetrakis(4-carboxyphenyl)porphyrinato]-Fe(III) chloride, referred to as TCPP(Fe), was used to successfully synthesize the ultrathin Zn-TCPP(Fe) nanosheets, which gave the lateral size of 1.1±0.2 μm and thickness of 7.4±2.9 nm (FIG. 5D and FIG. 6D).

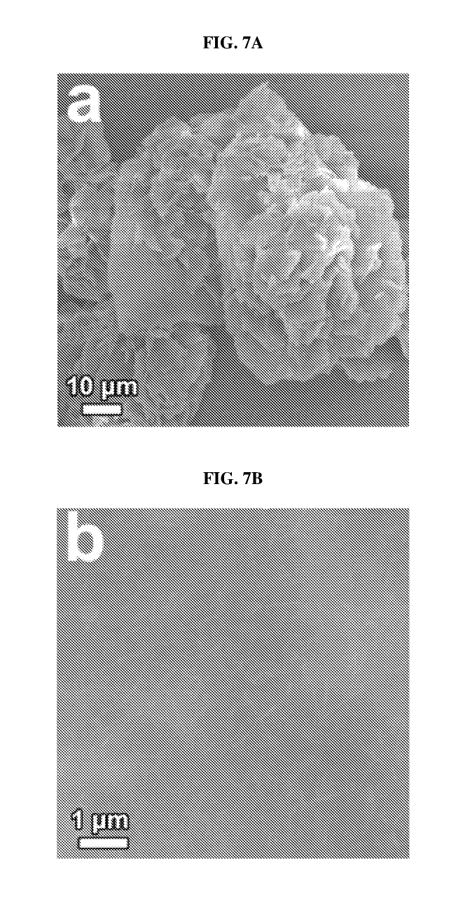

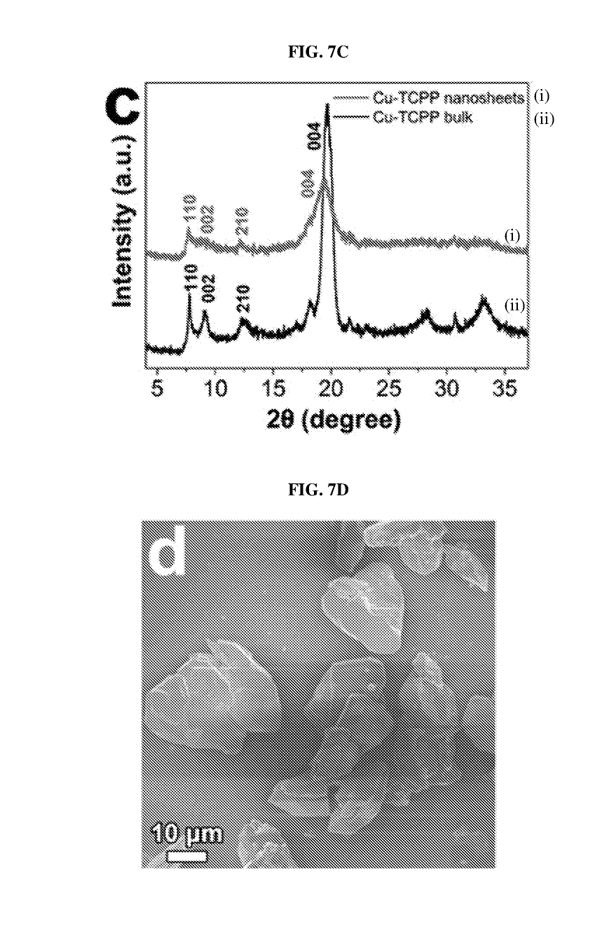

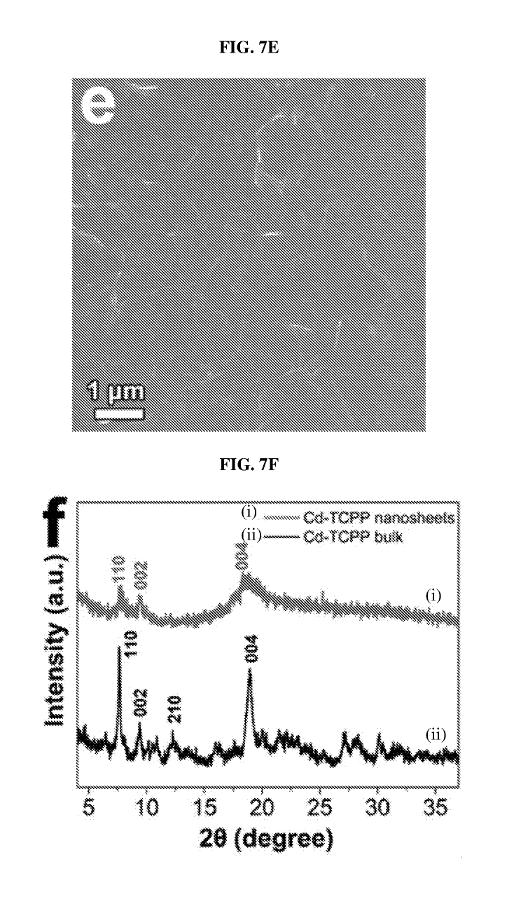

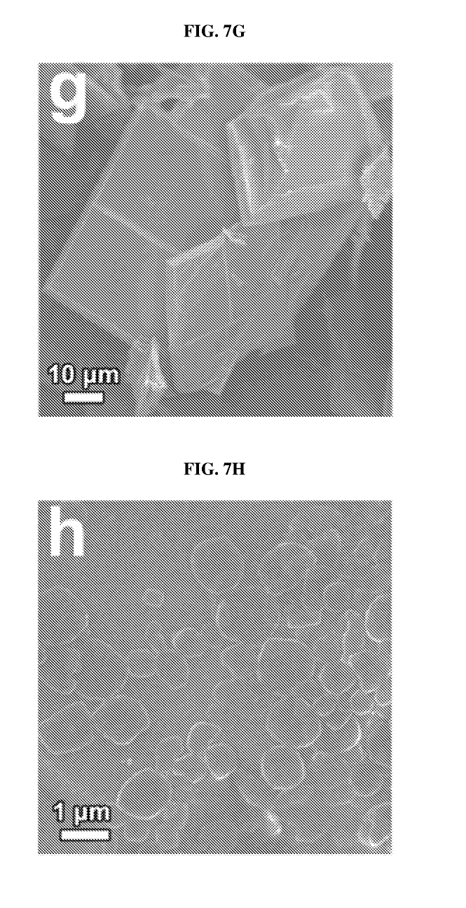

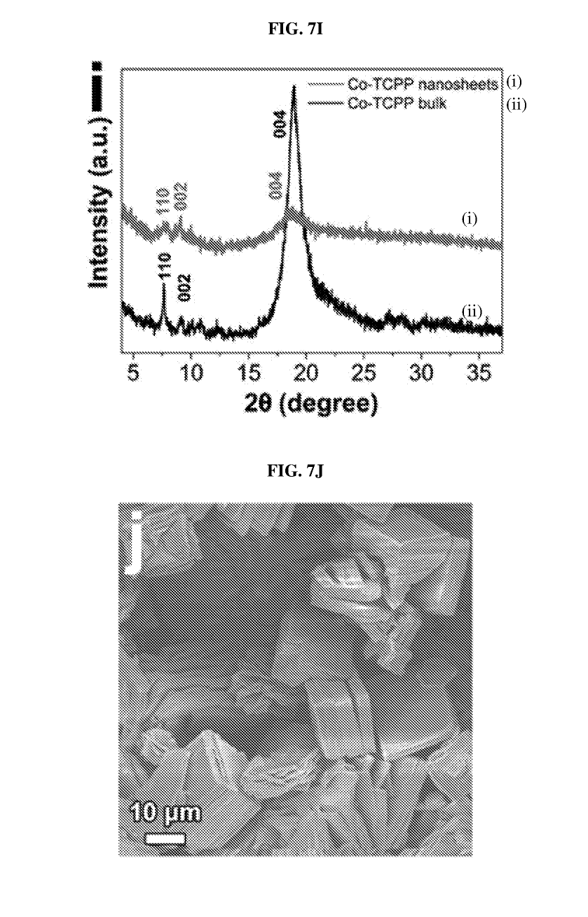

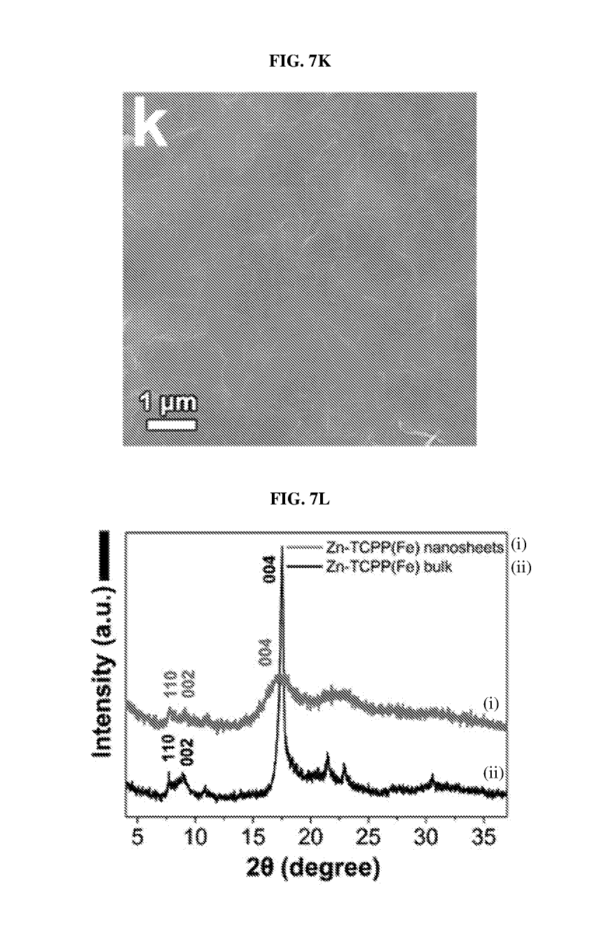

Similarly, Cu-TCPP(Fe) and Co-TCPP(Fe) nanosheets were also synthesized by the same method. The crystallinity of M-TCPP (M=Cu, Cd and Co) and M-TCPP(Fe) (M=Zn, Cu and Co) nanosheets were confirmed by TEM and the corresponding SAED and powder XRD measurements. The similar diffraction spots (insets in FIG. 5A to 5F) and XRD patterns (FIGS. 7C, 7F, 7I, and 7L) with those of Zn-TCPP nanosheets (FIG. 2F) were observed, indicating the crystal nature of these MOF nanosheets.

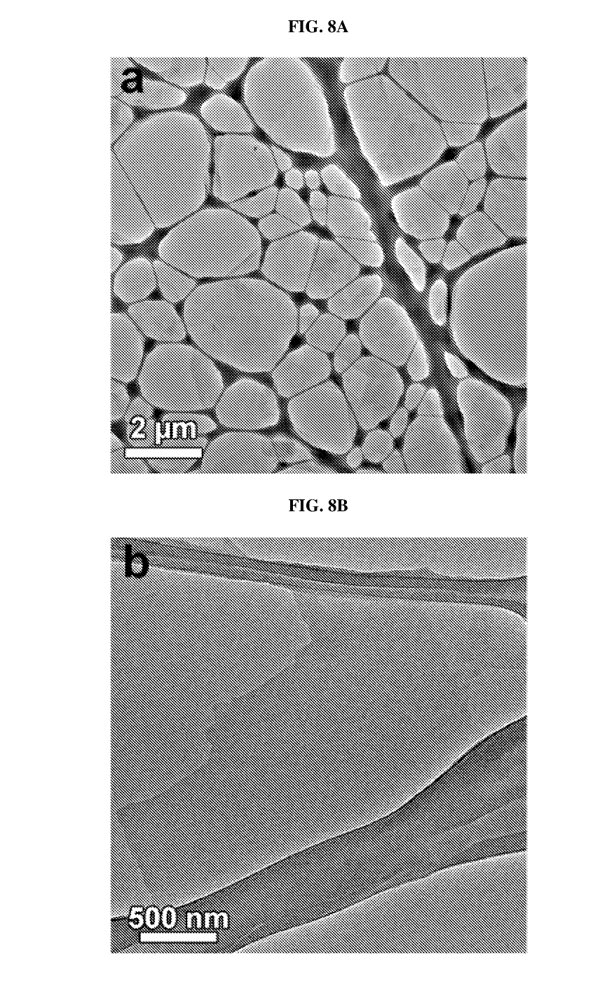

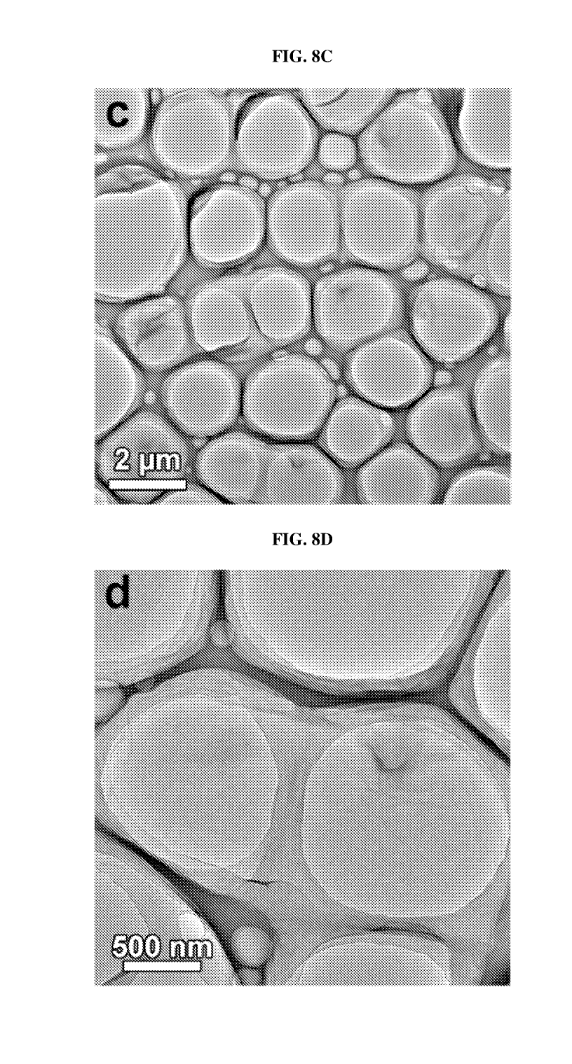

In addition, TCPP(Co), TCPP(Ni) and TCPP(Mn) were used instead of TCPP(Fe) to successfully synthesize the ultrathin 2D MOF nanosheets, such as Cu-TCPP(Co) and Cu-TCPP(Mn) nanosheets (FIG. 8A to 8D).

Besides divalent metal ions, such as Zn²⁺, Cu²⁺, Cd²⁺ and Co²⁺, trivalent metal ion, including but not limited to Al³⁺ can also be used for the synthesis of Al-TCPP and Al-TCPP(M′) (M′=Fe, Co, Mn and Ni) nanosheets.

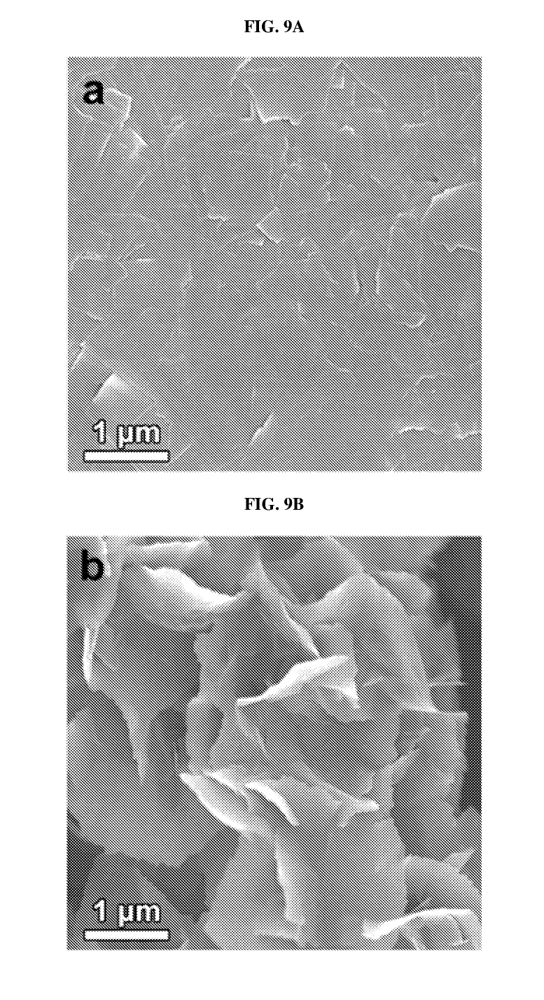

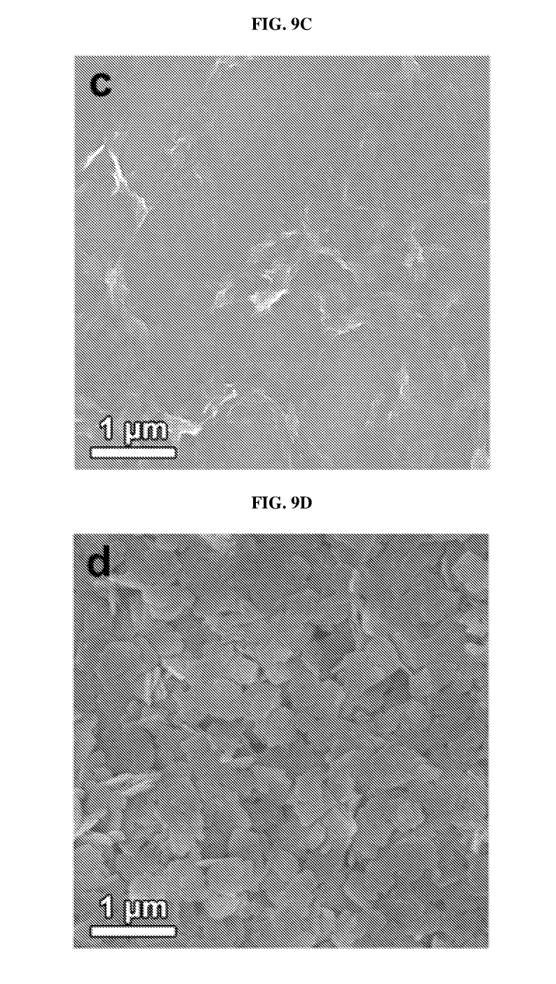

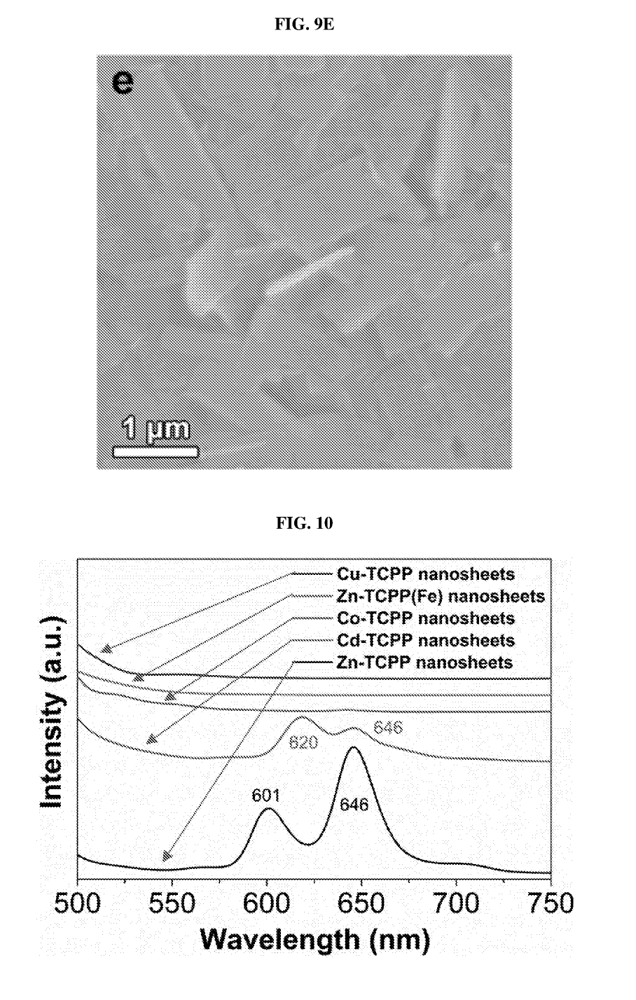

As shown in FIG. 9A, Al-TCPP with lateral size of about 1.0 μm and thickness of about 10 nm was obtained. Furthermore, terephthalic acid (BDC) and 2-aminoterephthalic acid (BDC-NH₂) were also used for the synthesis of 2D MOF nanosheets, such as Cu-BDC nanosheets (FIG. 9B), Cu-BDC-NH₂ nanosheets (FIG. 9C), Al-BDC nanosheets (FIG. 9D) and Al-BDC-NH₂ nanosheets (FIG. 9E). The aforementioned results prove that the surfactant-assisted synthetic method disclosed herein is simple, but universal, which can be used to tune the crystal growth kinetics of MOFs, resulting in the successful preparation of a series of freestanding ultrathin 2D MOF nanosheets.

### Example 8: Applications

### Example 8.1: DNA Detection

As known, 2D nanosheets, such as graphene and TMDs, with ultrathin thickness exhibit unique physical and chemical properties, enabling them as promising nanoplatforms for biosensing applications. Recently, the bulk MOFs have been used as sensors for the detection of small molecules, gas, volatile organic solvents and biomolecules.

As a proof-of-concept application 2D Cu-TCPP, Zn-TCPP(Fe) and Co-TCPP, with negligible fluorescence property compared to Zn-TCPP and Cd-TCPP nanosheets (FIG. 10), were used as new sensing platforms for DNA detection.

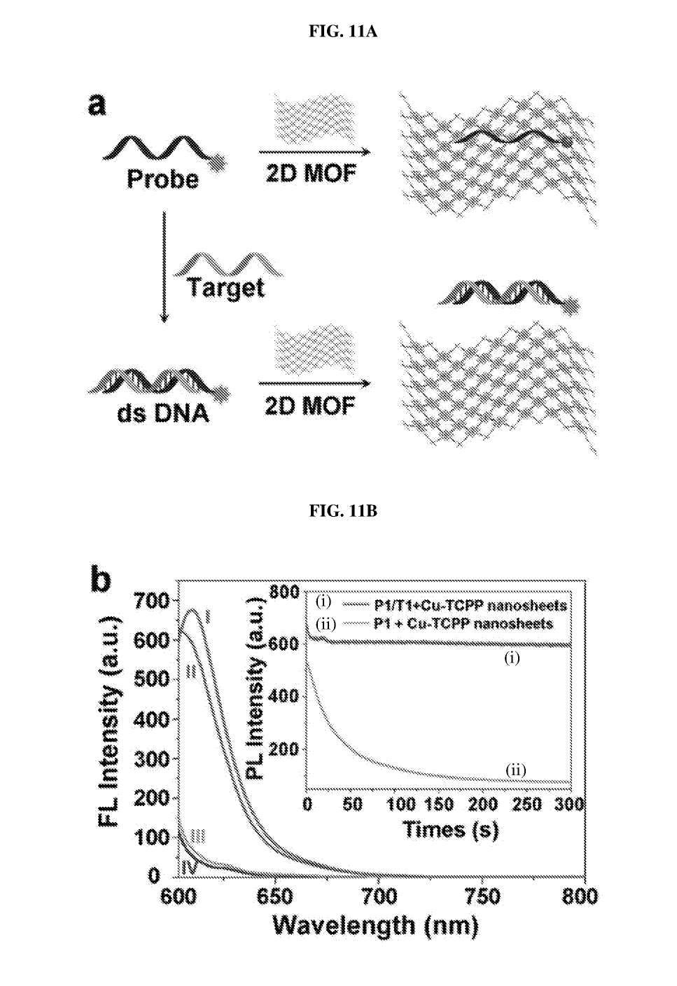

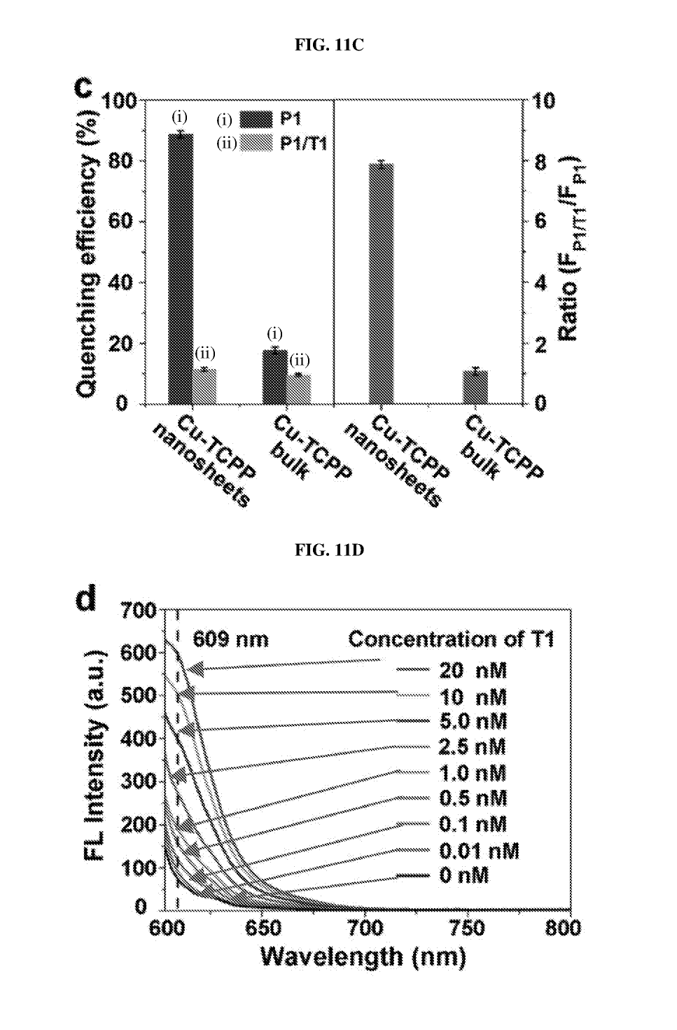

As shown in FIG. 11A, the TCPP ligand containing conjugated π-electron system allows for the binding of single-stranded DNA (ssDNA). Therefore, the dye-labeled ssDNA probe could be adsorbed on the basal surface of 2D MOF nanosheets, resulting in the fluorescence quenching of the dye through the fluorescence resoncance energy transfer (FRET). However, when the dye-labeled ssDNA probe is hybridized with its target complementary DNA to form double-stranded DNA (dsDNA), the interaction between the formed dsDNA and 2D MOF nanosheets becomes so weak that the dsDNA would detach from the surface of 2D MOF, resulting in the recovery of quenched fluorescence of dye, which is expected to provide quantitative detection of the target complementary DNA.

To validate the feasibility of the sensing strategy mentioned above, the following experiments were carried out. The DNA used in this work is listed in TABLE 1.

The Texas red-labeled ssDNA (denoted as P1) exhibits strong emission at the wavelength of 609 nm (FIG. 11B, curve I). After the addition of Cu-TCPP nanosheets, the fluorescence of P1 was quenched (FIG. 11B, curve III), resulting in the low fluorescence signal close to that of Cu-TCPP nanosheets (FIG. 11B, curve IV). The quenching kinetics was very fast, with quenching efficiency of up to 89% within 5 min after P1 was mixed with the solution of Cu-TCPP nanosheets (FIG. 11B, inset, origin curve), indicating the strong fluorescence quenching ability of 2D Cu-TCPP nanosheets. In contrast, after P1 was hybridized with the target complementary DNA, i.e. the Influenza A virus subtype H5N1 gene (denoted as T1) to form dsDNA, the fluorescence was greatly retained even in the presence of Cu-TCPP nanosheets (FIG. 11B, curve II, and green curve of inset).

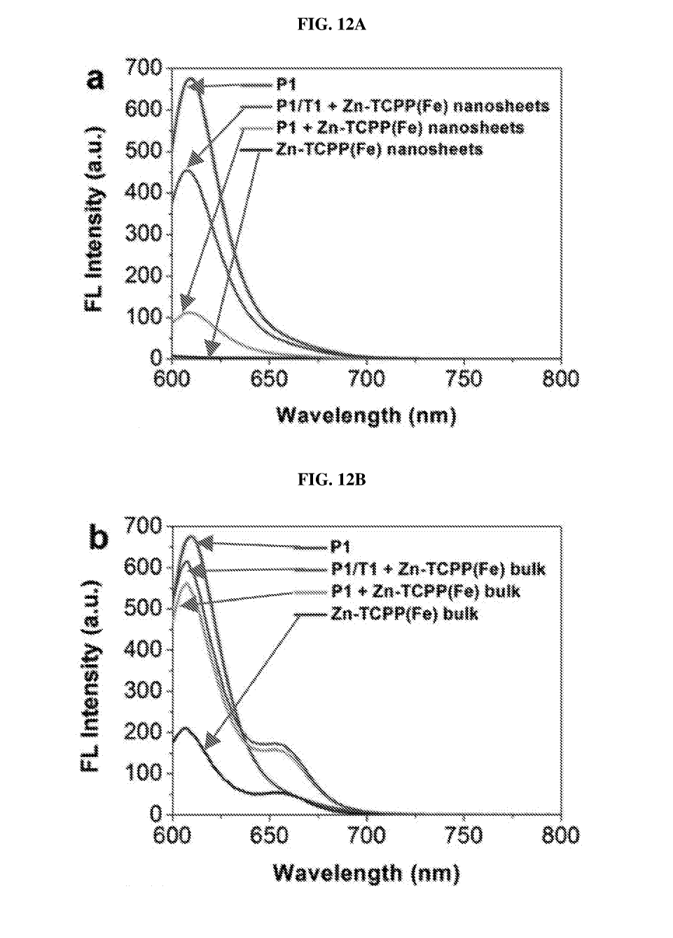

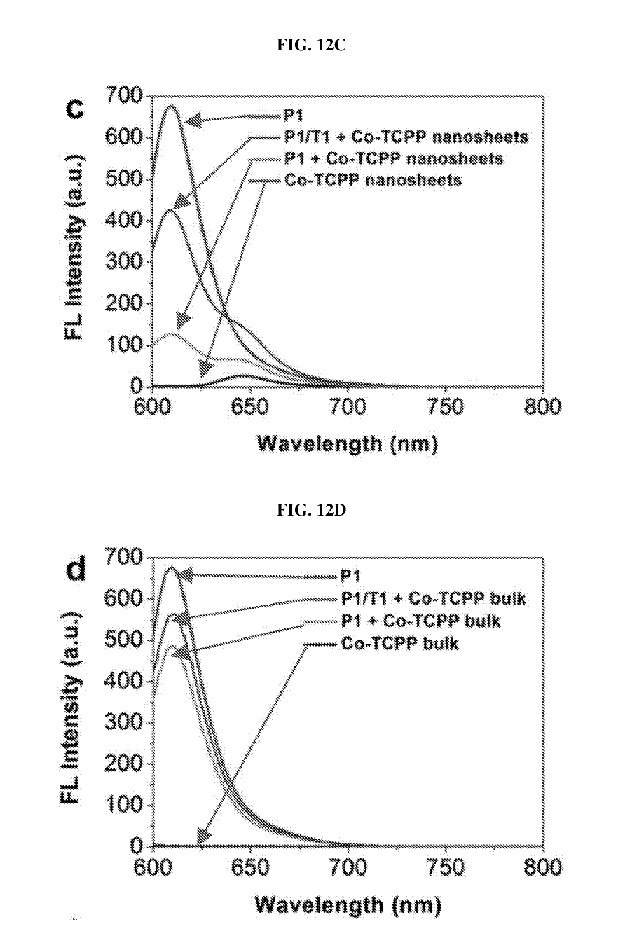

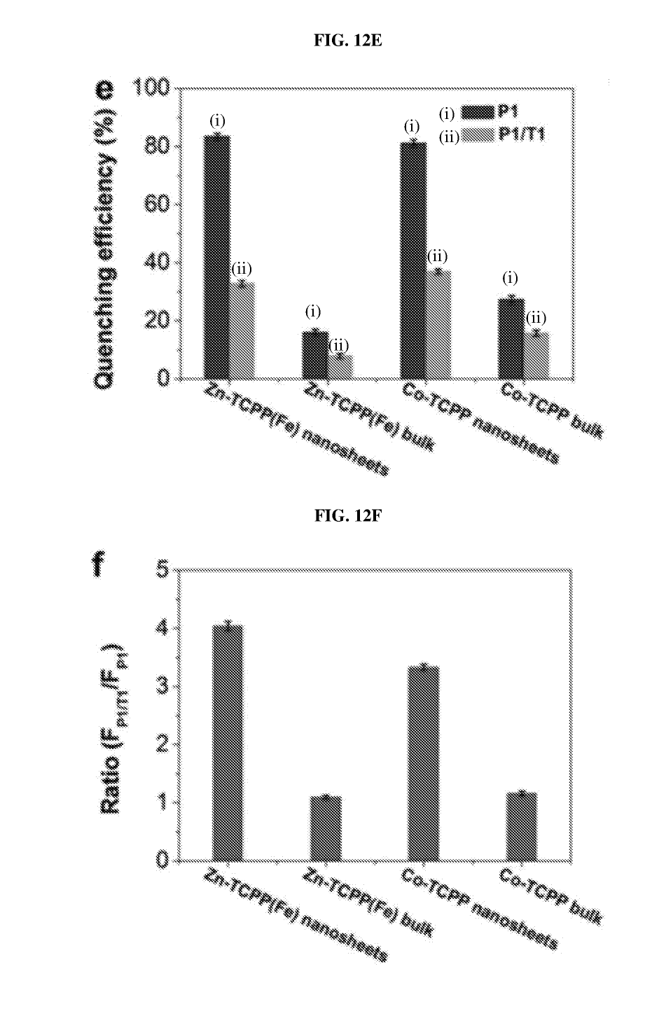

Compared to the bulk Cu-TCPP MOFs Cu-TCPP nanosheets showed much better fluorescence quenching ability and larger adsorption difference towards ssDNA and dsDNA. The quenching efficiencies of Cu-TCPP nanosheets and bulk Cu-TCPP MOFs for P1 are 89% and 19%, while for the P1/T1 duplex, the corresponding quenching efficiencies decrease to 11% and 10%, respectively (FIG. 11C, left panel). In addition, the obtained fluorescence intensity ratios (F_{P1/T1}/F_{P1}) are 7.9 and 1.1 for Cu-TCPP nanosheets and bulk Cu-TCPP MOFs, respectively (FIG. 11C, right panel). Such an enhancement in the fluorescence quenching ability from bulk material to nanosheet could be ascribed to the increased surface to volume ratio in nanosheets and more accessible active sites for energy/electron transfer from fluorescent molecules to quenchers. Similar to the Cu-TCPP MOF, the Zn-TCPP(Fe) and Co-TCPP MOFs also showed the same increased trend in the fluorescence quenching ability from bulk material to nanosheet (FIG. 12A to 12F).

Based on the aforementioned discussion, a bioassay for DNA detection based on 2D MOF nanosheets was established. Here, 2D Cu-TCPP nanosheet was chosen as the fluorescence quenching platform since it has better quenching efficiency and larger adsorption difference towards ssDNA and dsDNA compared to 2D Zn-TCPPP(Fe) and Co-TCPP nanosheets (FIG. 11C, FIGS. 12E and 12F). When the concentration of T1 increases, the retained fluorescence intensity of P1 also increases (FIG. 11D). The 2D Cu-TCPP nanosheet-based sensor shows a linear range between 0 and 5 nM with the detection limit of 20 pM for T1 (3σ), which is much lower compared to the previously reported fluorescent assays based on MOF particles.

Simultaneous detection of multiple targets in a homogeneous solution is significantly important in practical application for medical diagnosis, disease prevention, and environmental monitoring. Here, for the first time, a multiplexed DNA bioassay based on 2D MOF nanosheets was developed for the simultaneous detection of a virus for example the Influenza A virus subtype H5N1 gene (namely T1) and subtype H1N1 gene (denoted as T2), by using Texas-labeled ssDNA probe (namely P1) and TET labeled ssDNA probe (denoted as P2), respectively (TABLE 1).

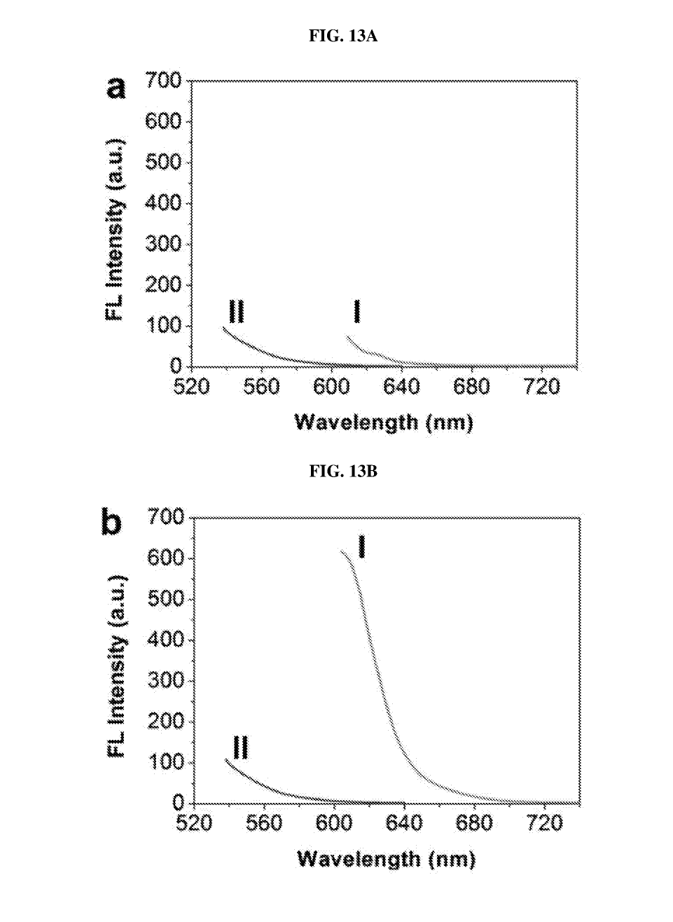

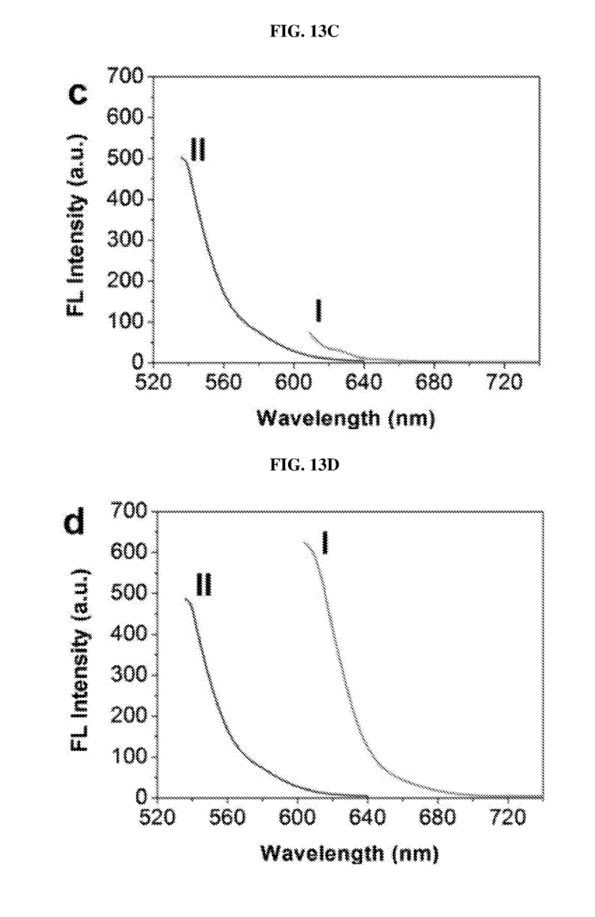

As shown in FIG. 13A, very low fluorescent signals were detected when the probes P1 and P2 mixed with 2D Cu-TCPP nanosheets. The introduction of the specific target DNA (T1 or T2) would selectively retain the fluorescent signal of the probe (P1 or P2) (FIGS. 13B and 13C). Moreover, after addition of the mixture of two target DNA (T1 and T2), both of the fluorescent signals of P1 and P2 could be retained (FIG. 13D), suggesting the feasibility of 2D MOF nanosheet-based fluorescent sensor for the simultaneous detection of multiple analytes.

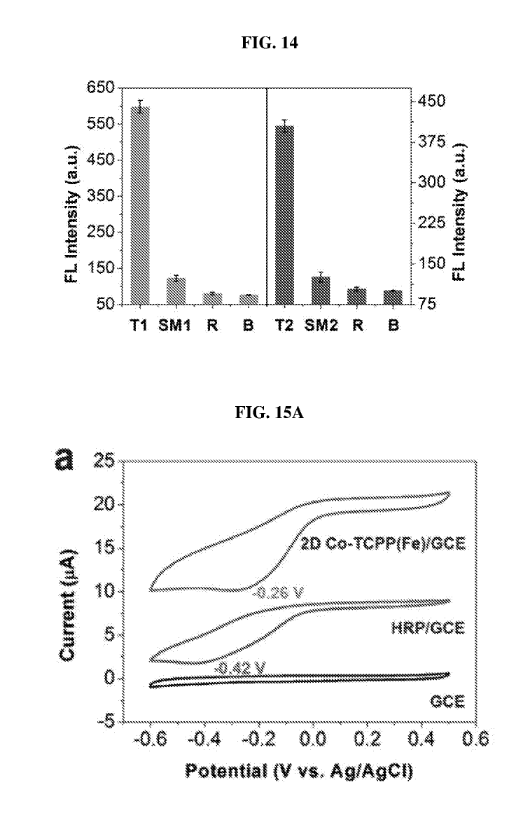

Furthermore, the selectivity of 2D Cu-TCPP nanosheet-based sensor was also evaluated by addition of single-base mismatch DNA (denoted as SM) and random DNA (denoted as R). As shown in FIG. 14, the SM and R did not cause the distinct increase of fluorescent signal. All these results demonstrate that the 2D Cu-TCPP nanosheet-based fluorescent sensor disclosed herein possessed the excellent selectivity, which may hold great promise for the real sample analysis.

The exemplifications above are intended to be illustrative, not limiting. Thus, it will be apparent to one skilled in the art that modifications may be made to the invention as described without departing from the scope of the invention.

### Example 8.2: Biomimetic Enzyme for Electrochemical Biosensing

Due to their similar structure (iron-porphyrin) with natural enzyme (peroxidase), the 2D MOF nanosheets including but not limited to (Co-TCPP(Fe), Cu-TCPP(Fe) and Zn-TCPP(Fe)) exhibit excellent catalytic activity as an peroxidase mimic. As a proof-of concept application, electrochemical sensor based on 2D Co-TCPP(Fe) nanosheets was developed for the detection of H₂O₂, which exhibit higher sensitivity and better stability than natural enzyme (HRP, horseradish peroxidase). Furthermore, the 2D Co-TCPP(Fe) based sensor was used for the real-time detection of H₂O₂ secretion by live cells due to its high sensitivity, fast response time, long-term stability and reproducibility.

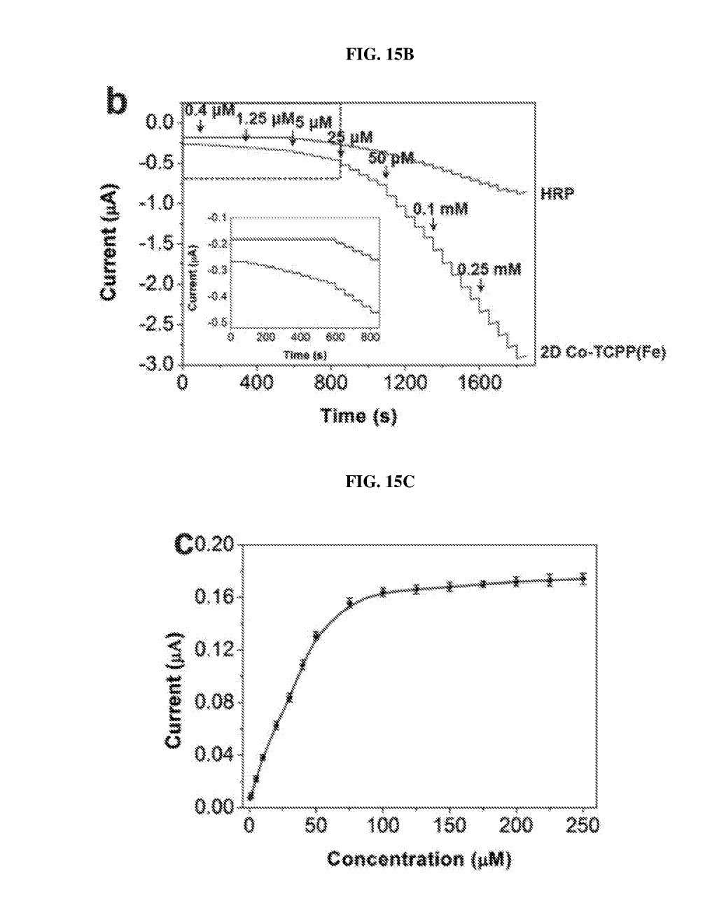

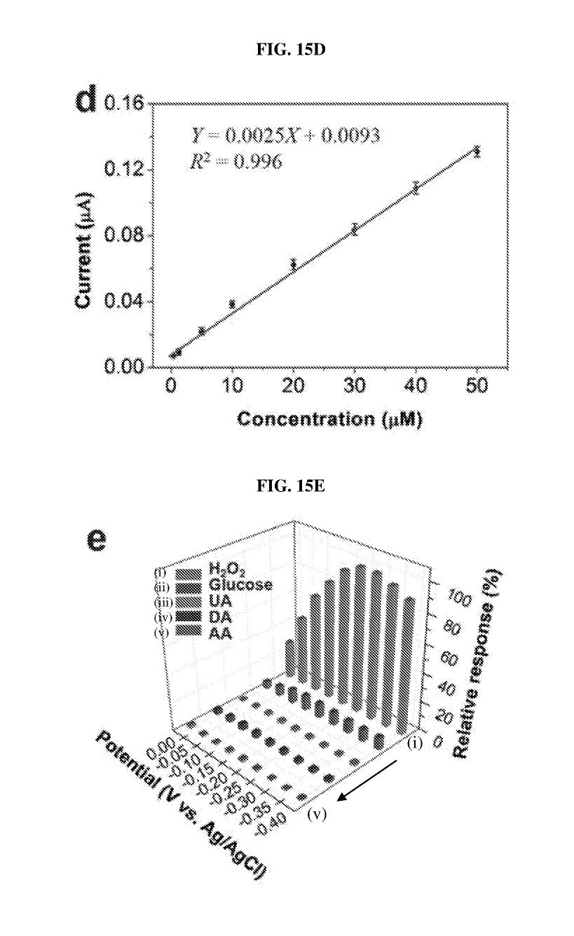

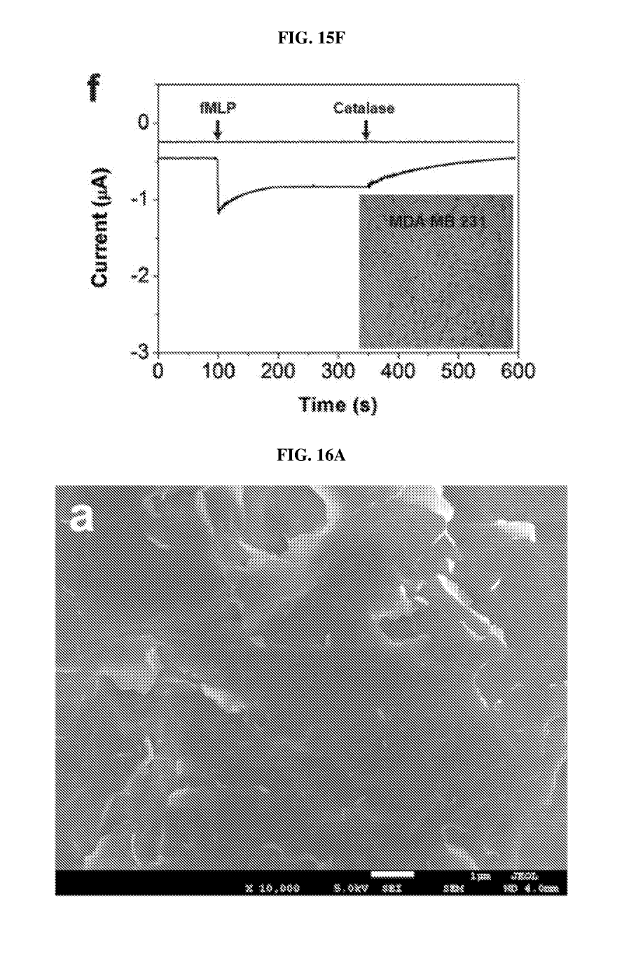

FIG. 15A shows CV curves of 2D Co-TCPP(Fe)/GCE (2D Co-TCPP nanosheets modified glass carbon electrode) with control electrodes such as HRP/GCE (horseradish peroxidase modified glass carbon electrode) and GCE (glass carbon electrode) in 0.1 M oxygen-free phosphate buffer solution (PBS, pH 7.4) containing 0.5 mM H₂O₂. 2D Co-TCPP(Fe)/GCE exhibits a well-defined reduction peak associated with the reduction of H₂O₂. The reduction peak potential of 2D Co-TCPP(Fe)/GCE is 150 mV more positive than that of HRP/GCE and the peak current is much larger as well, indicating that 2D Co-TCPP(Fe) nanosheets has a higher catalytic activity to the reduction of H₂O₂ than HRP. In contrast, no obvious reduction peak of H₂O₂ was detected in the same potential range for bare GCE.

FIG. 15B shows the typical amperometric responses of the 2D Co-TCPP(Fe)/GCE and HRP/GCE upon the successive injection of H₂O₂ into the PBS solution. Similar to the CV results, the response current of 2D Co-TCPP(Fe)/GCE was significantly higher than the one of HRP/GCE. Upon each addition of H₂O₂, the response of the 2D Co-TCPP(Fe)/GCE based-sensor rapidly reaches 95% of the steady-state value within 3 s. The good linear dependence on the H₂O₂ concentration in the range of 0.4-50 μM was achieved, with a detection limit down to 0.15 μM at a signal-to-noise (S/N) ratio of 3 (FIGS. 15C and 15D).

FIG. 15E shows the amperometric response of H₂O₂ and all the potential interfering substances (glucose, UA, DA and AA) at the 2D Co-TCPP(Fe)/GCE. The potential interfering substances do not cause any interference for the detection of H₂O₂, indicating the high selectivity of 2D Co-TCPP(Fe)/GCE toward H₂O₂ detection. Encouraged by the optimal performance, i.e., the broad linear dynamic range, low detection limit, high sensitivity and selectivity, of the 2D Co-TCPP(Fe)/GCE biosensor disclosed herein, the inventors used it for real-time tracking H₂O₂ secretion by live cells. The cells of 80% confluency (inset of FIG. 15F) were induced to release H₂O₂ by injecting N-formyl-methionyl-leucyl-phenylalanine (fMLP). Upon the addition of 10 μM fMLP, a significant increased current was observed at the 2D Co-TCPP(Fe)/GCE, followed by a gradual decrease of current, and reached a plateau after 60 s. Furthermore, the addition of catalase, which is a H₂O₂ scavenger, resulted in a sudden drop of current response followed by progressive decease down to the level of background signal afterwards. Control wells containing no breast cancer cells did not generate any signal response to the addition of fMLP or catalase. This observation substantially demonstrates that the H₂O₂ biosensor based on 2D Co-TCPP(Fe) nanosheets based electrode establishes a sensitive, reliable, and robust method for the routine determination of H₂O₂ secreted by live cells and could potentially be useful for further physiological and pathological investigations.

### Example 8.3: MOF Membrane for Separation

Separation with membranes is an energy-efficient and environmentally friendly alternative to absorptive separation processes. The most widely used membranes are polymer membranes, however, these membrane are subject to a trade-off between permeability and selectivity, known as Robeson's upper bound. Membranes based on porous materials, such as MOF nanosheets and COF nanosheets, were expected to overcome this limitation due to their highly ordered pores or channels with tunable size and shape.

Although mixed-matrix membranes made from polymer and MOF nanoparticles have been reported, to the best of the inventors' knowledge, there are no reports of membrane made of MOF nanosheets.

Due to the ultrathin nature of the 2D M-TCPP nanosheets with large lateral size (about 1 μm), MOF membrane were readily prepared using the ultrathin 2D MOF nanosheets as building block through suction filtration.

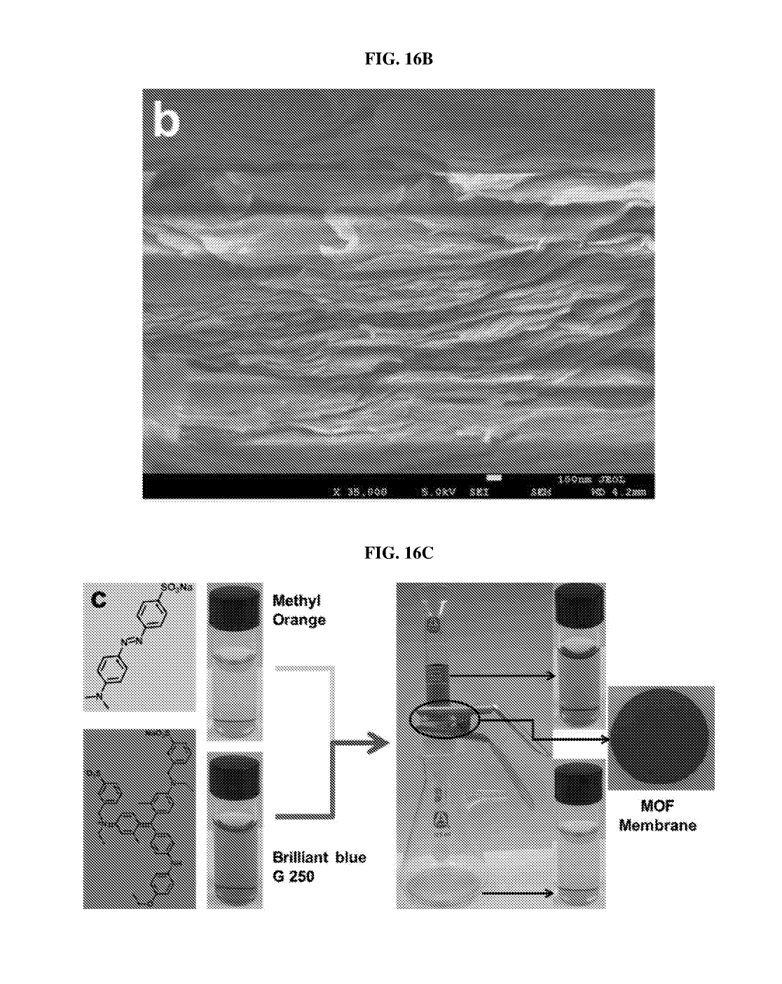

FIGS. 16A and 16B shows the SEM images of the membrane prepared from ultrathin Cu-TCPP nanosheets. As shown in FIG. 16A, the surface of MOF membrane was very similar with that of ultrathin 2D MOF nanosheets, the edge of MOF nanosheets were also observed duo to the restacking of Cu-TCPP nanosheets during formation of membrane. The corresponding cross-section SEM image (FIG. 16B) of the membrane clearly revealed the layered structure of the membrane prepared from ultrathin MOF nanosheets.

To demonstrate the possible separation property of MOF membrane, the as-prepared MOF membrane was employed to separate organic dye with different molecule size in aqueous solution (FIG. 16C). This MOF membrane successfully removed 20 PPM Brilliant blue G 250 from the aqueous mixture solution, while allowing the across of methyl orange. Dye removal from the solution is also confirmed by UV/Vis spectroscopy (λ=582 nm), which showed that more than 93% removal of the Brilliant blue G. Importantly, the MOF membrane was recycled after washing with methanol to remove the collected Brilliant blue G. The second run demonstrated more than 91% retention of the Brilliant blue G. These results indicating that beyond the simple removal of dyes from aqueous solutions, the MOF membrane achieved a separation of organic dyes with different molecule size.

In conclusion, for the first time, a facile surfactant-assisted synthetic method was reported to prepare a series of ultrathin 2D MOF nanosheets with thickness of sub-10 nm, such as M-TCPP (TCPP=tetrakis(4-carboxyphenyl)porphyrin, M=Zn, Cu, Cd, Co or Al) nanosheets, M-TCPP(M′) (M=Zn, Cu, Cd, Co and Al; M′=Fe, Co, Ni and Mn) nanosheets, M-BDC (BDC=terephthalic acid, M=Cu, Al) and M-BDC-NH₂ (BDC-NH₂=2-aminoterephthalic acid, M=Cu, Al) nanosheets. As a proof-of-concept application, some of the 2D M-TCPP nanosheets were successfully used as fluorescent sensing platforms for DNA detection, biomimetic enzyme for electrochemical biosensing and membrane for separation. Based on our experiments, the 2D Cu-TCPP nanosheet shows the best performance for sensing DNA with detection limit of 20 pM, which is much lower than the previously reported MOF particle-based fluorescent assays. In addition, the simultaneous detection of multiple DNA is also realized with our 2D Cu-TCPP nanosheets-based fluorescent sensor. Importantly, MOF membrane can also be prepared using ultrathin MOF nanosheets as buding blocks. This MOF membrane showed great separation property of dyes with different pore size.

### Example 9: MOF Heterostructures Based on 2D MOF Nanosheets

In addition, the inventors were also able to prepare MOF heterostructures based on 2D MOF nanosheets, i.e., 2D MOF nanosheets vertically growth on 1D MOF ribbons, rods and spheres, may also be prepared. MOF backbones with different morphology, such as ribbons, rods and spheres, were synthesized. Growth of 2D MOF nanosheets on the surface of these MOF backbones was then carried out.

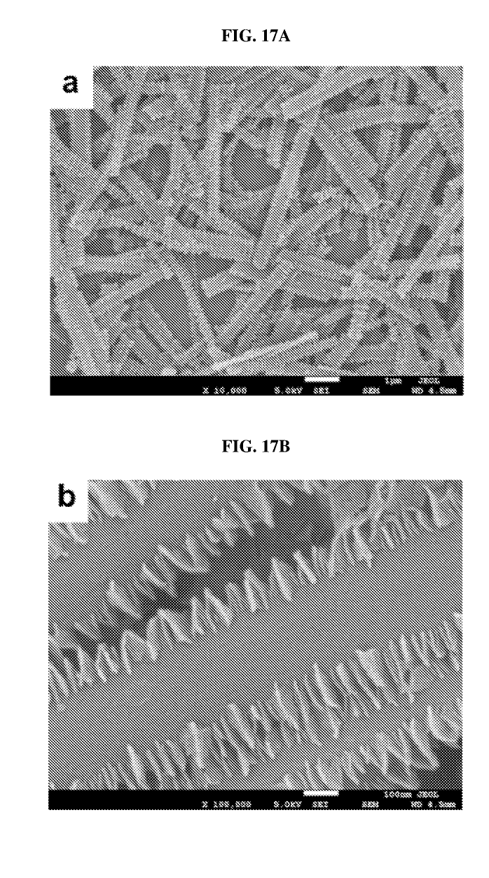

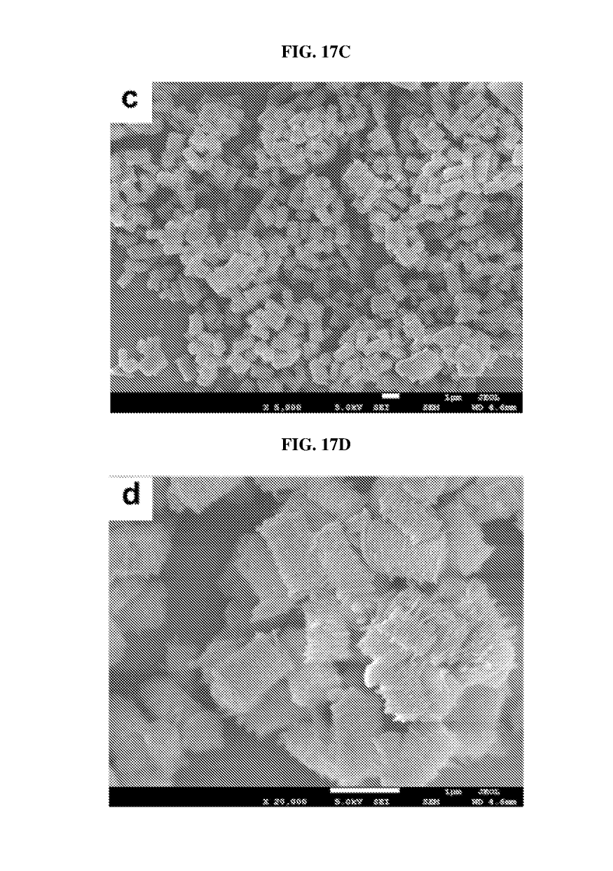

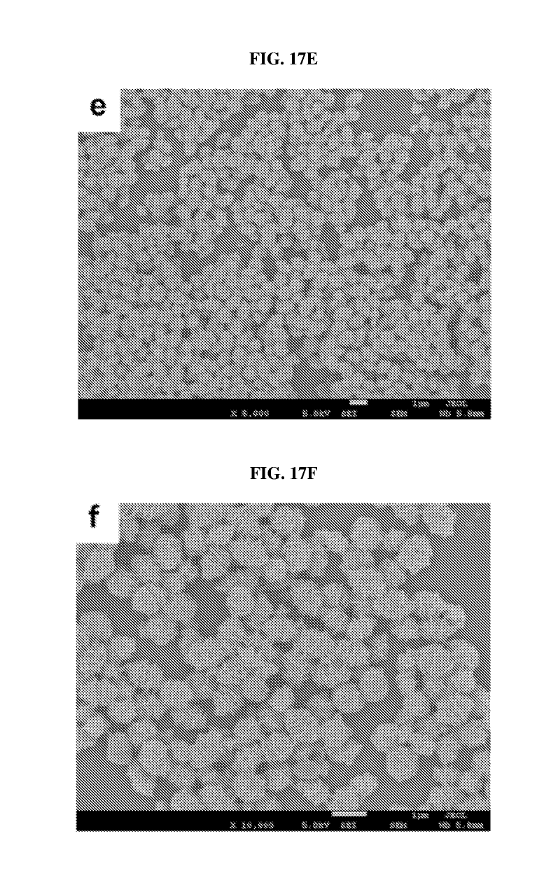

For example, Al-TCPP nanosheets with lateral size of about 100 nm were vertically growth on the surface of MI-69 ribbons (FIGS. 17A and 17B). In-TCPP nanosheets with lateral size of several hundred nanometers were vertically growth on the surface of MI-68 rods (FIGS. 17C and 17D). Zr-BTB nanosheets with lateral size of several hundred nanometers were vertically growth on the surface of MOF-525 spheres (FIGS. 17E and 17F). These results prove that the surfactant-assisted synthetic method disclosed herein is a kind of powerful method for preparation of a series of freestanding ultrathin 2D MOF nanosheets and heterostructures based on 2D MOF nanosheets.

### Example 10: Application in Biomimetic Catalysis

Inspired by the biological system, tremendous research efforts have been devoted to using synthetic systems to mimic natural enzymes with high catalytic activity and substrate selectivity. As known, in nature, metalloporphyrins play significant roles in many biological functions, including light harvesting, oxygen transportation and catalysis. However, it has been found that the aggregation of metalloporphyrins would obstruct their energetic characteristics as well as efficiencies in these applications. Many strategies have been demonstrated to overcome such drawbacks, and one of the most promising approaches is to encapsulate metalloporphyrins into metal-organic frameworks (MOFs) directly or to use metalloporphyrins as ligands to construct MOFs.

Herein, a series of water-stable metalloporphyrinic 2D MOF nanosheets with thickness of sub-10 nm are prepared by using surfactant-assisted synthetic method. Cu₂(COO)₄ paddle-wheel and TCPP(M) (M(III) tetrakis(4-carboxyphenyl)porphyrin) chloride, M=Fe, Co and Mn) are employed as metal nodes and ligands for the synthesis of 2D Cu-TCPP(M) nanosheets, respectively. As indicated in the catalytic studies, these 2D MOF nanosheets, especially 2D Cu-TCPP(Fe) nanosheets, with excellent substrate binding affinity and catalytic activity superior to HRP and other mimics, could serve as effective peroxidase mimic in aqueous media. Importantly, a hybrid nanosheets based on Cu-TCPP(Fe) and gold nanoparticles (Au NPs) were prepared by in situ growth of Au NPs on 2D Cu-TCPP(Fe) nanosheets. The obtained Au/Cu-TCPP(Fe) hybrid nanosheets can be used to mimic enzyme cascade reaction, in which the Au NPs and 2D Cu-TCPP(Fe) nanosheets possess intrinsic glucose oxidase (Gox)- and peroxidase-like activity, respectively.

2D Cu-TCPP(M) nanosheets with small diffusion barriers, high water stability and potential catalytically active centers meet the prerequisites for a biomimetic system. To test the catalytic activity, these 2D Cu-TCPP(M) nanosheets with different metalloporphyrins are used to catalyze the oxidation reaction of peroxidase substrate, 3,3′,5,5′-tetramethylbenzidine (TMB), which is widely used to evaluate the catalytic performance of enzyme mimic in the previous work.

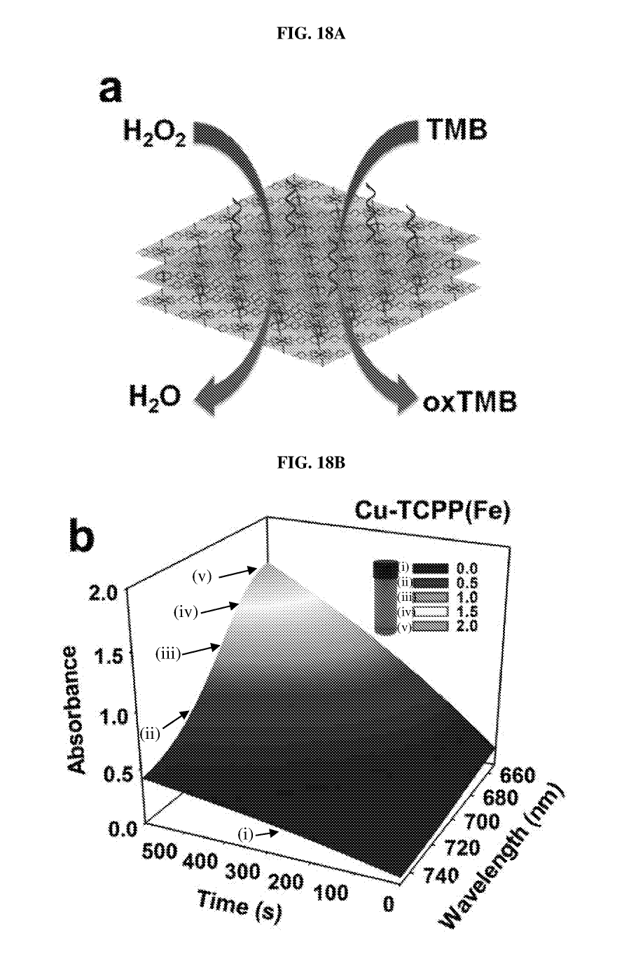

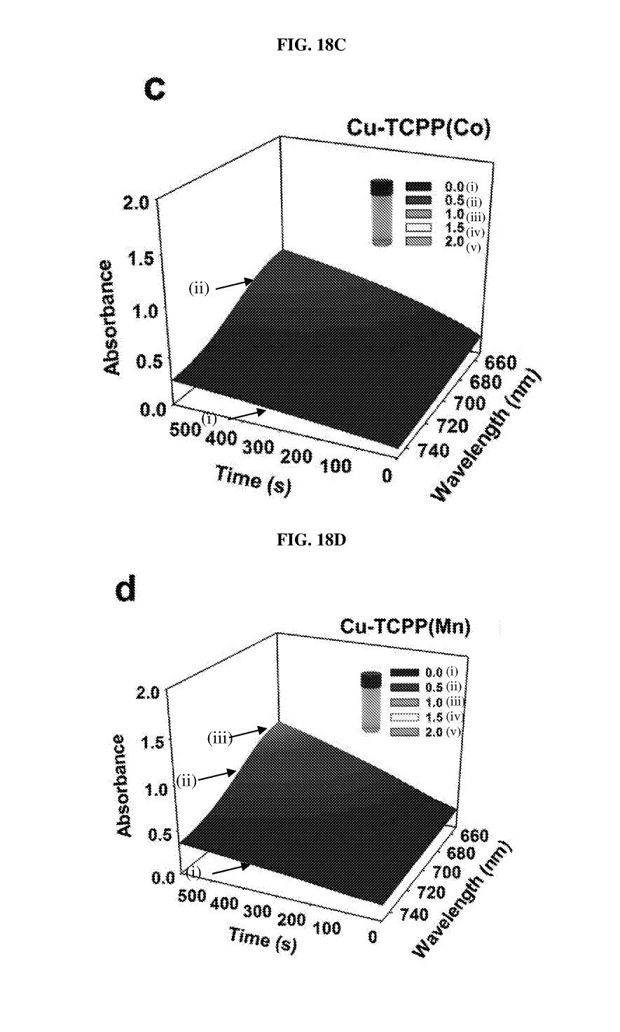

The schematic of this catalytic oxidation reaction is described in FIG. 18A, i.e., in the presence of H₂O₂, 2D Cu-TCPP(M) nanosheets can catalyze the oxidation of TMB to generate oxidized TMB (oxTMB) with blue color.

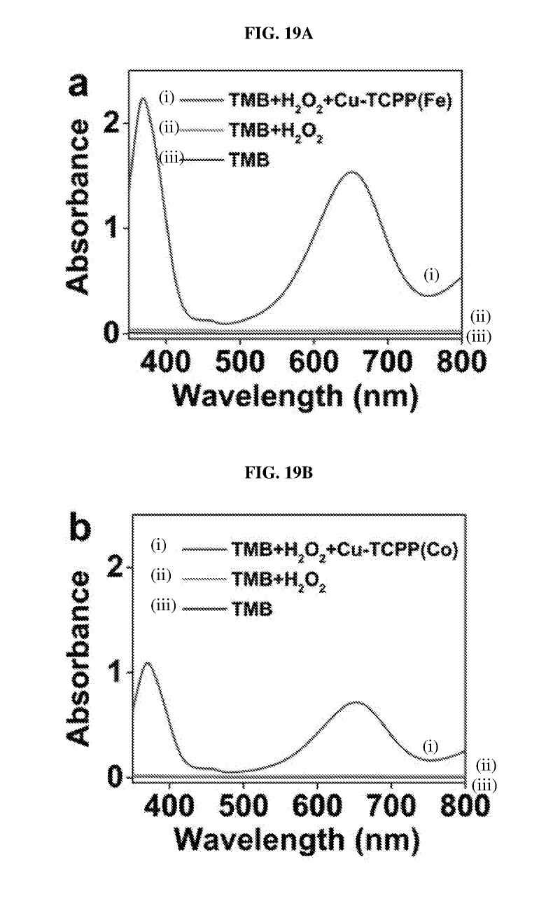

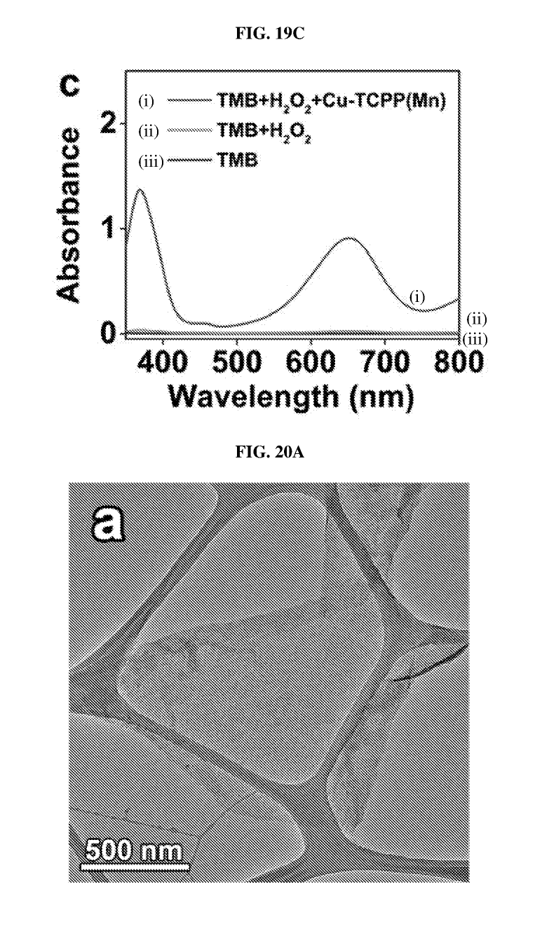

As shown in FIG. 19A to 19C, negligible absorption was observed without Cu-TCPP(M) nanosheets, suggesting that no oxidation reaction occurred in the absence of 2D Cu-TCPP(M) nanosheets. However, upon addition of 2D Cu-TCPP(M) nanosheets into the mixture of TMB and H₂O₂, the solutions showed intense characteristic absorbance at 369 nm and 652 nm after removing 2D Cu-TCPP(M) nanosheets, which could be attributed to the charge-transfer complexes derived from one-electron oxidation of TMB, confirming that the as-prepared 2D Cu-TCPP(M) nanosheets possess intrinsic peroxidase-like activity.

More importantly, the oxidation of TMB catalyzed by 2D Cu-TCPP(Fe) nanosheets produced a deep blue color in the solution with the maximum absorption peak at 652 nm (FIG. 18B), and the absorption peaks were less intensive by using 2D Cu-TCPP(Co) or Cu-TCPP(Mn) nanosheets as catalyst under the same condition (FIG. 18C, D), indicating that 2D Cu-TCPP(Fe) nanosheets exhibited higher catalytic activity than 2D Cu-TCPP(Co) and Cu-TCPP(Mn) nanosheets. The plausible reason may be that the as-prepared 2D Cu-TCPP(Fe) nanosheets were constructed with iron-porphyrin struts, which were the active centers of heme proteins.

In the control experiments, the catalytic activities of bulk Cu-TCPP(M) MOFs with the same concentration were also measured. As expected, the catalytic activities of bulk Cu-TCPP(M) MOFs toward the oxidation of TMB were lower than those of corresponding 2D Cu-TCPP(M) nanosheets, which could be attributed to the presence of more active sites with small diffusion barriers in ultrathin 2D MOF nanosheets.

Small Au NPs possess Gox-mimic activity, which can catalyze the oxidation of glucose to produce gluconic acid and H₂O₂ in the presence of oxygen. In addition, it has been demonstrated that 2D Cu-TCPP(M) nanosheets can act as effective peroxidase mimic. Therefore, an artificial enzymatic cascade system can be engineered based on 2D Cu-TCPP(M) supported Au NPs (Au/Cu-TCPP(M)).

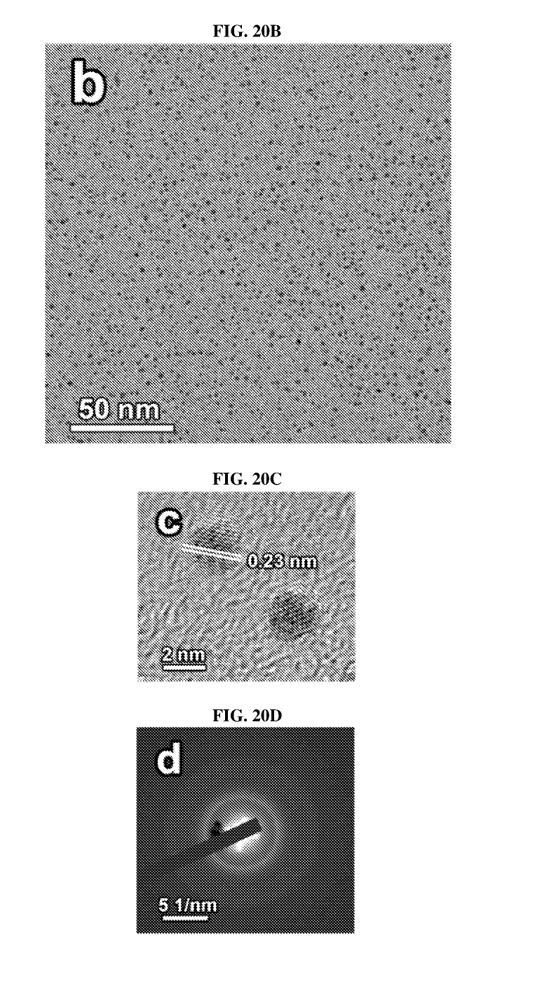

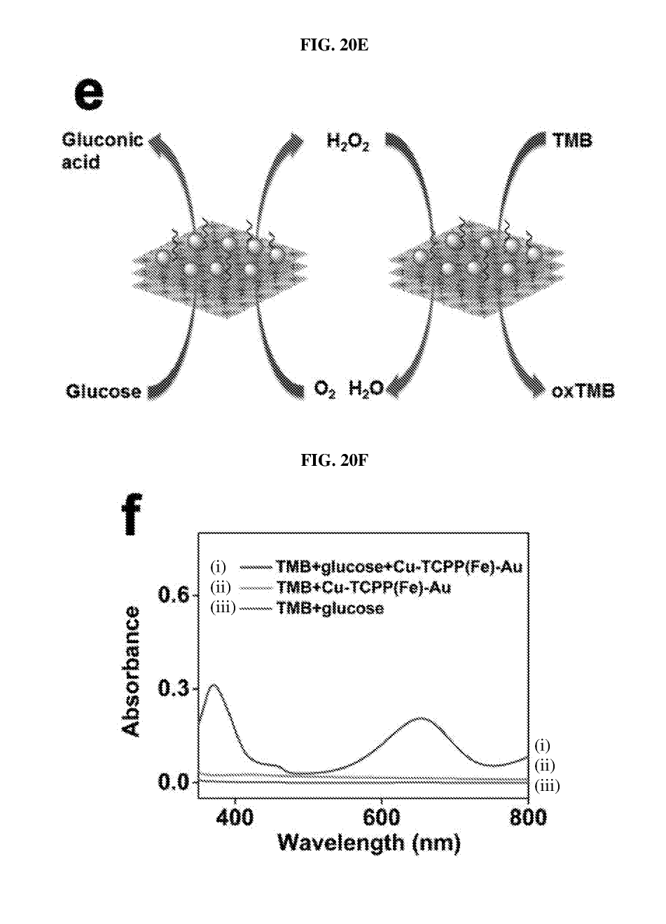

Taking 2D Au/Cu-TCPP(Fe) nanosheets as example, Au NPs were synthesized by in situ reduction of HauCl₄ with the presence of 2D Cu-TCPP(Fe) nanosheets. As shown in the TEM images in FIG. 20A and FIG. 20B, Au NPs with the size of 2-4 nm were uniformly deposited on the surface of 2D Cu-TCPP (Fe) nanosheets. The HRTEM image of Au NPs shows lattice spacing of 0.23 nm assignable to Au (111) plane (FIG. 20C). The SAED patterns further confirmed the high crystallinity of Au NPs (FIG. 20D).

Then the self-organized cascade reaction was investigated using Au/Cu-TCPP(Fe). The principle for the artificial enzymatic cascade system is shown in FIG. 20E. Au NPs can catalyze glucose to produce gluconic acid and H₂O₂ in the presence of 02. The in situ generated H₂O₂ can be used for oxidation of TMB catalyzed by Cu-TCPP(Fe).

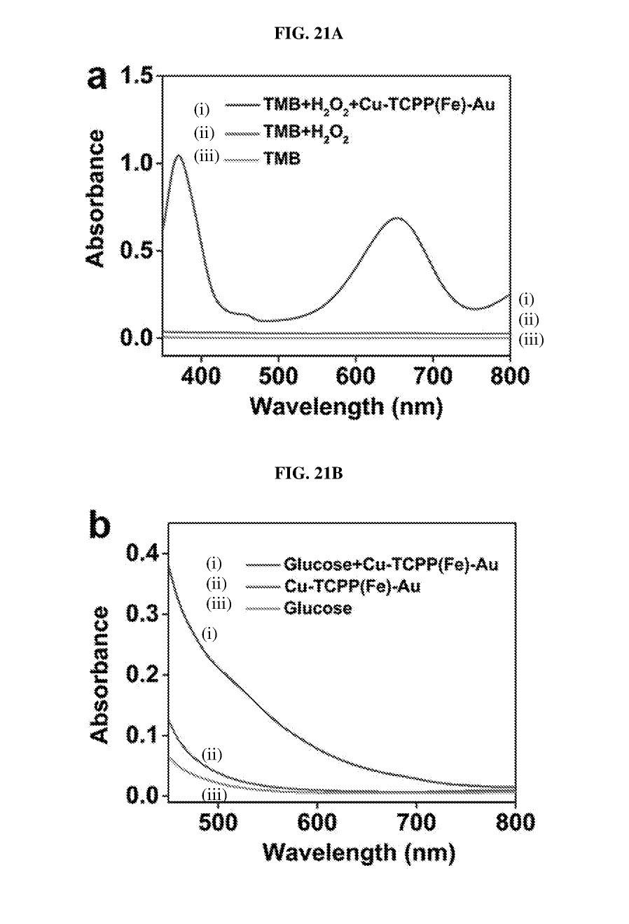

The peroxidase- and Gox-like activities of Au/Cu-TCPP(Fe) were investigated separately. It was found that Au/Cu-TCPP(Fe) can catalyze the oxidation of TMB in the presence of H₂O₂, which shows the major absorbance peaks at 369 and 652 nm (FIG. 21A). The result confirms that Au/Cu-TCPP(Fe) exhibits intrinsic peroxidase-mimic activity. In addition, Au/Cu-TCPP(Fe) can catalyze the oxidation of glucose in the presence of oxygen to produce gluconic acid and H₂O₂. The reaction solution is investigated with a gluconic acid-specific colorimetric assay, which shows a characteristic absorbance peak at 505 nm (FIG. 21B). The result confirm that Au/Cu-TCPP(Fe) exhibits the Gox-like activity. Therefore, Au/Cu-TCPP(Fe) can realize the self-organized cascade reaction, as shown in FIG. 20F, the major absorbance peaks at 369 and 652 nm can be observed due to the oxidation of TMB, demonstrating the cascade reaction has been carried out. Control experiments indicate that neither glucose nor Au/Cu-TCPP(Fe) alone in the presence of TMB can catalyze the cascade reaction (FIG. 20F).

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of preparing a metal-organic framework nanosheet, the method comprising
a) providing a mixture comprising a metal precursor, a ligand, and a surfactant by at least substantially dissolving the metal precursor, the ligand, and the surfactant in a suitable solvent, and
b) heating the mixture to obtain the metal-organic framework nanosheet.

2.-4. (canceled)

5. The method according to claim 1, wherein the ligand is selected from the group consisting of
(i) tetrakis(4-carboxyphenyl)porphyrin (TCPP),
(ii) TCPP(M′), wherein M′ is a metal different from the metal of the metal precursor and is selected from the group consisting of Fe, Co, Ni, Mn, and combinations thereof,
(iii) terephthalic acid (BDC),
(iv) 2-aminoterephthalic acid (BDC-NH₂),
(v) 2,6-naphthalenedicarboxylic acid,
(vi) 1,3,5-tris(4-carboxyphenyl)benzene (BTB), and
(vii) combinations of the above-mentioned.

6. The method according to claim 1, wherein the surfactant is selected from the group consisting of cetyltrimethylammonium bromide (CTAB), cetyltrimethylammonium chloride (CTAC), polyvinylpyrrolidone (PVP), polyethylene glycol (PEG), sodium dodecyl sulfate (SDS), and combinations thereof.

7. The method according to claim 1, wherein the solvent is selected from the group consisting of N,N-dimethylformamide (DMF), dimethylacetamide (DMA), N,N-diethylformamide (DEF), ethanol, methanol, water, and combinations thereof.

8. (canceled)

9. The method according to claim 1, wherein the metal of the metal precursor is a transition metal.

10. The method according to claim 9, wherein providing the mixture comprises adding the ligand to a solution comprising the metal precursor and the surfactant.

11. The method according to claim 10, wherein the ligand is dissolved in a mixture of N,N-dimethylformamide and ethanol with a volumetric ratio in the range from about 1:1 to about 4:1.

12. The method according to claim 10, wherein adding the ligand to the solution is carried out in a drop wise manner while the solution is being physically agitated.

13. The method according to claim 1, wherein the metal of the metal precursor is a Group 13 metal.

14. The method according to claim 13, wherein providing the mixture comprises adding the surfactant and the ligand to an aqueous solution comprising the metal precursor.

15. (canceled)

16. The method according to claim 14, wherein adding the surfactant and the ligand to the aqueous solution comprising the metal precursor is carried out in a drop wise manner while the aqueous solution comprising the metal precursor is being physically agitated.

17. The method according to claim 1, further comprising adding a crystallinity enhancing agent, a ligand dissolution enhancing agent, and/or a growth control agent to the mixture prior to heating.

18.-20. (canceled)

21. The method according to claim 1, wherein X at least substantially dissolving the metal precursor, the ligand, and the surfactant in a suitable solvent is carried out in the presence of a metal-organic framework nanostructured material.

22.-24. (canceled)

25. The method according to claim 1, wherein heating the mixture is carried out at a temperature in the range of about 60° C. to about 160° C.

26. (canceled)

27. The method according to claim 1, wherein lateral size of the metal-organic framework nanosheet is about 0.7 μm or more.

28. The method according to claim 1, wherein thickness of the metal-organic framework nanosheet is about 10 nm or less.

29. (canceled)

30. A metal-organic framework nanosheet having general formula (I)
M₁-L-M₂ (I),
wherein
M₁ is selected from the group consisting of zinc (Zn), copper (Cu), cadmium (Cd), cobalt (Co), zirconium (Zr), aluminum (Al), indium (In), and combinations thereof,
M₂ is nothing or selected from the group consisting of iron (Fe), cobalt (Co), nickel (Ni), manganese (Mn), and combinations thereof,
L is selected from the group consisting of tetrakis(4-carboxyphenyl)porphyrin (TCPP), terephthalic acid (BDC), 2-aminoterephthalic acid (BDC-NH₂), 2,6-naphthalenedicarboxylic acid, 1,3,5-tris(4-carboxyphenyl)benzene (BTB), and combinations thereof,
with the proviso that the metal-organic framework nanosheet is not copper 1,4-benzenedicarboxylate (Cu-BDC).

31. The metal-organic framework nanosheet according to claim 30, wherein thickness of the metal-organic framework nanosheet is less than about 10 nm.

32. The metal-organic framework nanosheet according to claim 30, wherein the metal-organic framework nanosheet is freestanding.

33.-36. (canceled)

37. A method of preparing a composite material comprising a metal-organic framework nanosheet and a noble metal nanoparticle, the method comprising
a) preparing a metal-organic framework nanosheet, comprising providing a mixture comprising a metal precursor, a ligand, and a surfactant by at least substantially dissolving the metal precursor, the ligand, and the surfactant in a suitable solvent, and heating the mixture to obtain the metal-organic framework nanosheet; or providing a metal-organic framework nanosheet having general formula (I)
M₁-L-M₂ (I),
wherein
M₁ is selected from the group consisting of zinc (Zn), copper (Cu), cadmium (Cd), cobalt (Co), zirconium (Zr) aluminum (Al), indium (In), and combinations thereof,
M₂ is nothing or selected from the group consisting of iron (Fe), cobalt (Co), nickel (Ni), manganese (Mn), and combinations thereof,
L is selected from the group consisting of tetrakis(4-carboxyphenyl)porphyrin (TCPP), terephthalic acid (BDC), 2-aminoterephthalic acid (BDC-NH₂), 2,6-naphthalenedicarboxylic acid, 1,3,5-tris(4-carboxyphenyl)benzene (BTB), and combinations thereof,
with the proviso that the metal-organic framework nanosheet is not copper 1,4-benzenedicarboxylate (Cu-BDC), and
b) dispersing the metal-organic framework nanosheet in an aqueous solution comprising a noble metal nanoparticle precursor and a reducing agent to obtain the composite material.

38.-41. (canceled)


SEQUENCE LISTING (7 sequences)

SEQ ID NO: 1 - DNA, 22 bp, Artificial Sequence
  Synthetic sequence H5N1 (T1)
catactgagaactcaagagtct

SEQ ID NO: 2 - DNA, 22 bp, Artificial Sequence
  Probe (P1) comprising Texas Red at 3′ end
  misc_feature (22)..(22): n is G conjugated to Texas Red
agactcttgagttctcagtatn

SEQ ID NO: 3 - DNA, 22 bp, Artificial Sequence
  Single-base mismatch DNA (SM1)
catattgagaactcaagagtct

SEQ ID NO: 4 - DNA, 22 bp, Artificial Sequence
  Synthetic sequence H1N1 (T2)
cgactacactctcgatgaagaa

SEQ ID NO: 5 - DNA, 22 bp, Artificial Sequence
  Probe (P2) comprising tetrafluororescein (TET) at 3′ end
  misc_feature (22)..(22): n is G conjugated to TET
ttcttcatcgagagtgtagtcn

SEQ ID NO: 6 - DNA, 22 bp, Artificial Sequence
  Single-base mismatch DNA (SM2)
cgactacactctggatgaagaa

SEQ ID NO: 7 - DNA, 22 bp, Artificial Sequence
  Random DNA (R)
tagcttatcagacagatgttga